(19) United States
(12) Patent Application Publication
(10) Pub. No.: US 2016/0339187 A1
(43) Pub. Date: Nov. 24, 2016

(54) DEVICES AND METHODS FOR CONTROLLED DRUG DELIVERY OF WET AEROSOLS

(71) Applicant: THE RESEARCH FOUNDATION FOR THE STATE UNIVERSITY OF NEW YORK, Albany, NY (US)

(72) Inventor: Gerald Smaldone, Setauket, NY (US)

(21) Appl. No.: 15/114,706

(22) PCT Filed: Jan. 23, 2015
(86) PCT No.: PCT/IB15/00896

§ 371 Date: Jul. 27, 2016

Related U.S. Application Data
(60) Provisional application No. 61/934,564, filed on Jan. 31, 2014.
(60) Provisional application No. 61/989,637, filed on May 7, 2014.

(51) Int. Cl. A61M 11/00; A61M 11/06; A61M 15/00

(52) U.S. Cl. CPC A61M 11/003; A61M 15/0086; A61M 15/0016; A61M 15/0018; A61M 15/0021; A61M 11/06; A61M 15/0091; A61M 11/005; A61M 15/0085

(57) ABSTRACT

The invention relates to the field of aerosolization by wet nebulizers, and in particular aerosols made by vibrating membranes. A capture chamber moderates the aerosol particle distributions with the primary effect on the larger particles.

### FIELD OF THE INVENTION

The invention relates to the field of aerosolization by wet nebulizers, and in particular aerosols made by vibrating membranes.

### BACKGROUND

Aerosols generated from wet nebulizers are difficult to control. For example the most modern systems include ultrasonic mesh systems. Compared to conventional jet nebulizers, they are very efficient usually nebulizing over 80% of the nebulizer charge. However, the aerosols generated have several problems including: a significant component of large particles in the aerosol distribution which causes deposition of the drug in the throat, poor quality control of the overall aerosol distribution, difficulty in controlling breathing pattern which affects deposition in the lungs and difficulty in controlling device output (i.e. inhaled mass) to the patient. What is needed is a device and method that mitigates these issues without sophisticated electronics.

### SUMMARY OF THE INVENTION

The invention relates to the field of aerosolization by wet nebulizers, and in particular aerosols made by vibrating membranes. Methods and devices are described that control particle size, flow and delivery of aerosols, in order to achieve the highest regional lung deposition (e.g. 100-87%) with the lowest possible upper airway deposition (e.g. 0-13%) and maximal total lung deposition (respirable mass).

In one embodiment, the present invention contemplates an aerosol capture device comprising: a) an opening configured to connect to an aerosol generator, b) a chamber configured to capture all emitted aerosol particles from an aerosol generator when an operating aerosol generator is connected to said opening, said chamber comprising a top and bottom, said bottom in fluid communication with said opening, said top comprising a one-way valve in fluid communication with, c) inhalation and exhalation openings, said inhalation opening comprising a mouthpiece. In one embodiment, the mouthpiece comprises a tongue bar. In one embodiment, the device further comprises a narrowing tube or stenosis connected to said chamber at said inhalation opening, e.g. addition of a stenosis in the distal tubing of the chamber. In a preferred embodiment, the narrowing tube or stenosis has an obstruction or baffle positioned in the inner diameter (e.g. to deflect and/or restrain the flow or air and aerosolized particles). In one embodiment, the obstruction or baffle rises up from the bottom or drops down from the top of the narrowing tube or stenosis. In one embodiment, the obstruction or baffle extends into the inner diameter as far as the radius of the inner diameter.

The present invention also contemplates an embodiment of an aerosol capture device comprising: a) an opening configured to connect to an aerosol generator comprising a vibrating mesh, said mesh comprising holes of less than 5.0 microns in diameter, b) a chamber configured to capture all emitted aerosol particles, and at least a portion contact the chamber, from an aerosol generator when an operating aerosol generator is connected to said opening, said chamber comprising a top and bottom, said bottom in fluid communication with said opening, said top comprising a one-way valve in fluid communication with, c) inhalation and exhalation openings, said inhalation opening comprising a mouthpiece. Preferably, the mesh hole size is less than or equal to 4.0 microns in diameter, more preferably less than or equal to 3.5 microns in diameter, and most preferably less than 3.4 microns in diameter, but larger than 1.5 microns in diameter. In one embodiment, the device further comprises a narrowing tube or stenosis connected to said chamber at said inhalation opening. In one embodiment, said narrowing tube comprises an obstruction or baffle positioned therein.

In one embodiment, a single chamber is contemplated. For example, said device lacks other chambers, such as a dosing chamber. In one embodiment, the chamber is attached to a narrowing tube or stenosis positioned at the end of the chamber opposite the aerosol generator.

It is not intended that the present invention be limited by the shape of the chamber, which can be square, rectangular, spherical and the like. In one embodiment, said chamber is tubular in shape. It is also not intended that the present invention be limited by the shape or dimensions of the narrowing tube or stenosis. The tube geometry can be varied as needed. In one embodiment, the tube is between 60 and 80 millimeters long, more preferably between 70 and 75 millimeters long (e.g. 72 mm). In one embodiment, the narrowing tube has an outer diameter of between 20 and 30 millimeters, more preferably between 20 and 25 millimeters, and most preferably between 21 and 23 millimeters (e.g. 22 millimeters), with an inner diameter of between 16 and 20 millimeters, more preferably between 17 and 19 millimeters (e.g. 18 millimeters).

It is also not intended that the present invention be limited by the composition of the chamber, i.e. the materials used to make it. However, in a preferred embodiment said chamber comprises anti-static plastic. It is also preferred that the narrowing tube be made of anti-static plastic, although other materials can be used.

It is also not intended that the present invention be limited by the size of volume of the chamber. In one embodiment, the chamber has a volume of between 10 and 250 milliliters, more preferably between 50 and 150 milliliters. In one embodiment, the volume is 90 milliliters. In one embodiment, the volume is 170 milliliters.

It is not intended that the present invention be limited by the nature of the aerosol generator. In one embodiment, the aerosol generator is a jet nebulizer. However, in one embodiment, the generator comprises a vibrating nebulizer.

In one embodiment, the present invention contemplates a method of capturing aerosol, comprising: 1) providing i) an aerosol generator, and ii) an aerosol capture device, said device comprising: a) an opening configured to connect to said aerosol generator, b) a chamber configured to capture all emitted aerosol particles from an aerosol generator when an operating aerosol generator is connected to said opening, said chamber comprising a top and bottom, said bottom in fluid communication with said opening, said top comprising a one-way valve in fluid communication with, c) inhalation and exhalation openings, said inhalation opening comprising a mouthpiece; 2) connecting said aerosol generator to said aerosol capture device through said opening; and 3) operating said aerosol generator under conditions such that said chamber capture all emitted aerosol particles from said aerosol generator. Again, the generator can be of a variety of types, including a jet nebulizer. However, in one embodiment, said aerosol generator comprises a vibrating nebulizer, such as an ultrasonic membrane nebulizer. In one embodiment, the chamber is connected to a narrowing tube or stenosis positioned at said inhalation opening between said chamber and said mouthpiece. In one embodiment, said narrowing tube comprises an obstruction positioned therein.

In one embodiment, the present invention contemplates a method of capturing aerosol, comprising: 1) providing i) an aerosol generator, and ii) an aerosol capture device, said device comprising: a) an opening configured to connect to said aerosol generator, b) a chamber configured to capture all emitted aerosol particles from an aerosol generator when an operating aerosol generator is connected to said opening, said chamber comprising a top and bottom, said bottom in fluid communication with said opening, said top comprising a one-way valve in fluid communication with, c) inhalation and exhalation openings, said inhalation opening comprising a mouthpiece; 2) connecting said aerosol generator to said aerosol capture device through said opening; and 3) operating said aerosol generator under conditions such that said chamber capture all emitted aerosol particles from said aerosol generator, wherein at least a portion of said particles contact said chamber, and said particles are mixed with air so as to reduce particle size such that the majority of aerosol particles are less than 2.5 microns in diameter. In one embodiment, said chamber is connected to a narrowing tube or stenosis positioned at said inhalation opening. In one embodiment, the narrowing tube contains an obstruction or baffle that projects into the lumen of the narrowing tube.

In one embodiment, the present invention contemplates an apparatus comprising an aerosol generator (reversibly or irreversibly) attached to an aerosol capture device, said device comprising a) an opening connected to and in fluid communication with said aerosol generator, b) a chamber configured to capture all emitted aerosol particles from said aerosol generator when said aerosol generator is operating, said chamber comprising a top and bottom, said bottom in fluid communication with said opening, said top comprising a one-way valve in fluid communication with, c) inhalation and exhalation openings, said inhalation opening comprising a mouthpiece. Again, the chamber can be of various shapes and types. In one embodiment, said chamber comprises anti-static plastic. Again, the generator can be selected among various types, including a jet nebulizer. However, in one embodiment, said aerosol generator comprises a vibrating membrane. In one embodiment, the apparatus further comprises a narrowing tube or stenosis connected to said chamber at said inhalation opening. In one embodiment, the narrowing tube contains an obstruction or baffle that projects into the lumen of the narrowing tube.

In one embodiment, the present invention contemplates an apparatus comprising an aerosol generator attached to an aerosol capture device, said aerosol generator comprising a vibrating mesh, said mesh comprising holes of less than 5.0 microns in diameter, said capture device comprising a) an opening connected to and in fluid communication with said aerosol generator, b) a chamber configured to capture all emitted aerosol particles from said aerosol generator when said aerosol generator is operating, said chamber comprising a top and bottom, said bottom in fluid communication with said opening, said top comprising a one-way valve in fluid communication with, c) inhalation and exhalation openings, said inhalation opening comprising a mouthpiece. Preferably, the mesh hole size is less than or equal to 4.0 microns in diameter, more preferably less than or equal to 3.5 microns in diameter, and most preferably less than 3.4 microns in diameter, but larger than 1.5 microns in diameter. In one embodiment, the apparatus further comprises a narrowing tube or stenosis connected to said chamber at said inhalation opening.

In one embodiment, the present invention contemplates a method of administrating an aerosol, comprising: a) providing, to an inhaling and exhaling subject, an aerosol generator attached to an aerosol capture device, said device comprising a) an opening connected to and in fluid communication with said aerosol generator, b) a chamber configured to capture all emitted aerosol particles from said aerosol generator when said aerosol generator is operating, said chamber comprising a top and bottom, said bottom in fluid communication with said opening, said top comprising a one-way valve in fluid communication with, c) inhalation and exhalation openings, said inhalation opening comprising a mouthpiece, said subject contacting said mouthpiece; and b) activating said aerosol generator under conditions wherein i) said chamber captures all emitted aerosol particles from said aerosol generator, ii) at least a portion of said aerosol particles leave said chamber when said subject inhales on said mouthpiece, iii) said one-way valve blocks gases from entering said top of said chamber when said subject exhales, thereby directing said gases through said exhalation opening. In one embodiment, the mouthpiece comprises a tongue bar and said subject contacts said tongue bar with said subject's tongue. In one embodiment, said chamber comprises anti-static plastic. Again, a number of different aerosol generators can be employed, including a jet nebulizer. In one embodiment, said aerosol generator comprises a vibrating nebulizer, such as an ultrasonic membrane nebulizer, wherein there is a vibrating membrane. In one embodiment, the generator comprises a fluid reservoir, e.g for containing the drug to be delivered. In one embodiment, a narrowing tube or stenosis is connected to said chamber at said inhalation opening.

In one embodiment, the present invention contemplates a method of administrating an aerosol, comprising: a) providing, to an inhaling and exhaling subject, an aerosol generator attached to an aerosol capture device, said aerosol generator comprising a vibrating mesh, said mesh comprising holes of less than 5.0 microns in diameter, said capture device comprising a) an opening connected to and in fluid communication with said aerosol generator, b) a chamber configured to capture all emitted aerosol particles from said aerosol generator when said aerosol generator is operating, said chamber comprising a top and bottom, said bottom in fluid communication with said opening, said top comprising a one-way valve in fluid communication with, c) inhalation and exhalation openings, said inhalation opening comprising a mouthpiece, said subject contacting said mouthpiece; and b) activating said aerosol generator under conditions wherein i) said chamber captures all emitted aerosol particles from said aerosol generator, and mixes said particles with air, ii) at least a portion of said aerosol particles contact said chamber, iii) at least a portion of said aerosol particles leave said chamber when said subject inhales on said mouthpiece, iii) said one-way valve blocks gases from entering said top of said chamber when said subject exhales, thereby directing said gases through said exhalation opening. Preferably, the mesh hole size is less than or equal to 4.0 microns in diameter, more preferably less than or equal to 3.5 microns in diameter, and most preferably less than 3.4 microns in diameter, but larger than 1.5 microns in diameter.

In one embodiment, the nebulizer runs continuously so breath actuation is not needed. The chamber captures all particles and holds them until the patient inhales. Inspiratory flow can be controlled via inspiratory resistances. During this time the aerosol is “conditioned”, that is there is partial evaporation and at least some of the larger particles, in particular, get smaller. One or more valves at the mouthpiece prevent backflow of gases during expiration.

In one embodiment, the present invention contemplates an apparatus comprising an aerosol generator comprising a vibrating element, said vibrating element located at the entrance of a chamber, said chamber configured to capture all emitted aerosol particles from said aerosol generator when said aerosol generator is operating, said chamber comprising an exit, said exit comprising a one-way valve in fluid communication with, c) inhalation and exhalation openings, said inhalation opening comprising a mouthpiece. In one embodiment, there is no connector piece; rather the generator is directly attached (whether reversibly or irreversibly) to the chamber. In one embodiment, said vibrating element serves as the floor of the chamber. In one embodiment, said chamber comprises anti-static plastic. In one embodiment, the mesh is incorporated in chamber base with no opening for airflow, all inspiratory gases enter the chamber via one-way orifice, the chamber volume can be reduced (e.g. 90 mL) and the valve system designed to accommodate different breathing patterns. Preferably, the mesh hole size is less than or equal to 4.0 microns in diameter, more preferably less than or equal to 3.5 microns in diameter, and most preferably less than 3.4 microns in diameter, but larger than 1.5 microns in diameter. In one embodiment, the apparatus further comprises a narrowing tube or stenosis connected to said chamber at said inhalation opening.

In one embodiment, the present invention contemplates an apparatus comprising an aerosol generator comprising a vibrating element, said vibrating element located at the entrance of a chamber and comprising mesh, said mesh comprising holes less than 5.0 microns in diameter, said chamber configured to capture all emitted aerosol particles from said aerosol generator when said aerosol generator is operating, said chamber comprising an exit, said exit comprising a one-way valve in fluid communication with, c) inhalation and exhalation openings, said inhalation opening comprising a mouthpiece. Preferably, the mesh hole size is less than or equal to 4.0 microns in diameter, more preferably less than or equal to 3.5 microns in diameter, and most preferably less than 3.4 microns in diameter, but larger than 1.5 microns in diameter. In one embodiment, the apparatus further comprises a narrowing tube or stenosis connected to said chamber at said inhalation opening.

In one embodiment, the present invention contemplates a method of administrating an aerosol, comprising: a) providing an aerosol capture device, said capture device comprising a) an aerosol generator comprising a vibrating mesh, said mesh comprising holes of less than 4.0 microns in diameter, said aerosol generator positioned on the floor of b) a chamber configured to capture all emitted aerosol particles from said aerosol generator when said aerosol generator is operating, said chamber comprising a top, sides and said floor, said top comprising a one-way valve in fluid communication with c) at least one opening for contacting a subject, said floor comprising d) an opening for introducing air into said chamber; and b) activating said aerosol generator under conditions wherein i) said chamber captures all emitted aerosol particles from said aerosol generator, and mixes said particles with air, ii) at least a portion of said aerosol particles contact said chamber, iii) at least a portion of said aerosol particles leave said chamber when said subject inhales on said mouthpiece, and iii) said one-way valve blocks gases from entering said top of said chamber when said subject exhales. It is preferred that said mixing of said particles with air reduces the particle sizes of a plurality of particles. It is preferred that said mixing of said particles with air reduces the particle sizes such that the majority of aerosol particles are less than 2.5 microns in diameter. It is preferred that the particles are reduced in size by said chamber or impact on said chamber. In one embodiment, a narrowing tube or stenosis is connected to said chamber at said inhalation opening.

The chamber acts to retain particles that would otherwise be lost by exhalation and to modify them by various mechanisms to make the final inhaled distribution more respirable, e.g. bypassing the upper airways favoring deposition in the lungs. These mechanisms include mixing with room air and shrinkage and impaction on the walls. Other mechanisms include impaction on baffles in the chamber including the inspiratory/expiratory connections and valves and modifications to the chamber that favor chamber deposition of the larger particles. We have evidence for these chamber deposition processes in scintigraphy scans of the chamber demonstrating deposition on the wall, the valves and the connectors. They show deposition (1) at connectors-entrance effects (2) at the valves (3) on the walls. These all add up and can be manipulated to increase impaction when desired. For example, additional baffles can be added to increase impaction when desired.

It is not intended that the present invention be limited to the nature of the drug(s) aerosolized with the various embodiments discussed herein. In one embodiment, the drug is an antibiotic or mixture of antibiotics. In one embodiment, the drug is interferon.

### DEFINITIONS

Inhaled Mass (IM) is the amount of nebulized aerosol captured in vitro that theoretically reaches the mouth of a patient. To measure IM we quantified radioactivity following nebulization on the T piece, cascade impactor (including stages and housing) and IM filter using a calibrated ratemeter (Linak, Denmark). IM was reported as a percent of the initial nebulizer charge. Radioactive deposition in the prototype chamber was also measured with the ratemeter. The sum of all components represents the total mass balance, which should approximate 100% of the nebulizer charge, barring aerosol losses to the environment.

We define treatment time as that needed to completely nebulize a known volume. For experimental work, the known volume was 0.5 mL of radiolabeled saline. We measured treatment times for both breathing patterns (as discussed below) and continuous and breath actuated nebulization.

Particle distributions were also measured without simulated breathing, the so-called “standing cloud.”

“Wet nebulizers” include all forms of wet nebulization, such as jet nebulizers, vibrating membranes, vibrating crystals and vibrating wafers.

### DESCRIPTION OF THE INVENTION

The invention relates to the field of aerosolization by wet nebulizers, and in particular aerosols made by vibrating membranes. Methods and devices are described that control particle size, flow and delivery of aerosols. Vibrating systems can be improved if (1) simple, non-software methods are employed to prevent expiratory losses of aerosol, (2) the treatment time is reduced (e.g. compared to breath actuated systems) (3) inhaled particle distributions are less variable between devices and (4) the particle distributions contain fewer large particles (e.g. fewer particles larger than 3.5 microns, and more preferably fewer particles larger than 3 microns, and most preferably, fewer particles larger than 2.5 microns). Herein, we demonstrate the value of a holding chamber and valve system designed to capture generated particles, retain them during expiration and present them on demand to a patient. The chamber also conditions the particles and provides an aerosol in the range defined by our laboratory as truly respirable (approx. ≦2.5 μm in diameter). Finally, our system does not require the use of breath actuation resulting in a reduction in treatment time.

Vibrating membrane nebulizers generate aerosols efficiently but tend to produce large particles outside the respirable range. Using a holding chamber such as shown here promotes mixing of particles with room air allowing conditioning of the aerosol resulting in an increase in the respirable fraction (RF). The increase in RF combined with the retention of particles that would be lost during expiration significantly increases the respirable mass preserving much of the inherent efficiency of the nebulizer but minimizing upper airway deposition.

Some investigators have designed chamber systems to improve device efficiency. For example, Vecellio and colleagues developed the Idehaler (LaDiffusion Technique Francaise, Saint Etienne, France), a chamber designed to increase inhaled mass. Their group has reported drug delivery in human studies using the Aeroneb nebulizer (Aerogen, USA). While they have reported significant increases in delivery to the lungs, the particle distributions appear unchanged and the device is designed primarily to capture the plume of the Aeroneb device. Nektar has used a similar device for the delivery of antibiotics in spontaneously breathing patients with reports of lung deposition averaging 43% in normal subjects. The combination of high inhaled mass (reported by Vecellio et al, in vitro (approx. 90%)) and the relatively low average lung deposition reported by Corkery et al at Nektar is consistent with significant upper airway deposition.

Our chamber captures particles that would be lost during exhalation but, unlike other designs, it modifies the component of the distribution that is destined to deposit in the upper airways. Our data suggest that there are two important phenomena affecting particles in our chamber; first, the effects of ventilating wet aerosols with room air results in shrinkage, second, there is impaction of particles in the chamber. The evaporation effects are best shown in FIGS. 18-20. More specifically, the standing cloud distribution (FIG. 18) shifts to the left with the effects of ventilation (FIG. 19). Using the chamber results in the final distributions seen in FIG. 20. The latter effect is likely due to impaction of large particles in the chamber. Further proof of this is suggested in FIG. 21 where only minimal effects are seen with added humidity.

While ventilating with room air will shrink the particles (as with all wet aerosols), the chamber is necessary to preserve efficiency and take out the remaining large particles as demonstrated by the significant increase in respirable mass over that seen with spontaneous breathing without the chamber.

Future designs of aerosol delivery systems can be further optimized. First, while chamber design can moderate the distributions of a population of mesh devices, limiting the population of meshes produced to those with holes smaller than those of Omron #3 (e.g. Omron 1 and 2) will help ensure that the final conditioned distributions approach that of the AeroTech II. Our data indicate that, compared to breath actuation, treatment time can be reduced with a chamber but meshes that produce particles that are too small (e.g. <Omron 1) will effectively prolong treatment time with no gain in deposition.

Controlling the flow of room air into the chamber is important in finalizing the aerosol distribution and lung deposition. These principles are illustrated in FIG. 15, a drawing of an idealized chamber based on our experience to date. The mesh is inherent to the chamber and does not require fittings dependent on airflow through the mesh (unlike that of the Omron device used in our experiments, which could not be sealed). Inspiration can be regulated via a valved one-way inspiratory orifice, which provides some resistance to flow (to control patient inspiration) and helps define the flow of air into the chamber. With the base of the device sealed, leaks are eliminated, inspiratory/expiratory valve design is simplified and chamber volume can be reduced.

It is preferred that, when using the chamber with an aerosol generator, there is a reliably low residual (15% or less) and respirable mass is maximized. Best results will be achieved when there is minimal mesh variation (e.g. hole size is constant from device to device) and the mesh is easily replaceable (even by the patient). In one embodiment, the holding chamber permits continuous breathing, allowing for a shorter treatment time (versus breath actuated administration).

### BRIEF DESCRIPTION OF THE DRAWINGS

FIG. 1 is a schematic of a testing system for evaluating the results of using a holding chamber in the manner described herein. The schematic shows: 1) a vibrating nebulizer containing a solution to be aerosolized, 2) a valved holding chamber with antistatic properties and a low resistance flap valve, 3) a piston ventilator that mimics a patient breathing with different breathing patterns, 4) a cascade impactor to measure particle distribution, 5) a filter to capture particles that are not captured by the cascade.

FIG. 2 is a bar graph showing results in terms of the radioactivity added to the nebulizer (a mass balance). Data are shown for 3 Omron devices. The left panel represents slow and deep breathing, with a rapid expiration; while the right panel represents a more rapid pattern consistent with a patient with COPD (low inspiratory volume prolonged expiration higher breathing frequency.

FIG. 3 shows four graphs depicting actual particle distributions. The slow and deep pattern data is shown on the left and the COPD pattern on the right. Open circles are distributions without the holding chamber, filled circles are with the holding chamber.

FIG. 4 is a schematic of a bench testing setup for determining inhaled mass and particle distribution for a jet nebulizer for both ‘standing cloud’ (no ventilation) and during breathing.

FIG. 5 shows inhaled mass and nebulizer residuals as percentages of the nebulizer charge for three devices: Omron, Pan and Sidestream.

FIG. 6 is a graph where aerosols from 3 different jet nebulizers sampled by cascade impaction are plotted on log probability paper: green dots Misty-Neb, red dots AeroEclipse, black dots AeroTech II.

FIG. 7 is a deposition image of a patient following inhalation during tidal breathing from the Misty-Neb jet nebulizer.

FIG. 8 is a deposition image from the same patient following inhalation of interferon aerosol from the AeroEclipse jet nebulizer.

FIG. 9, is a Gamma camera image from another patient following inhalation of pentamidine aerosol during tidal breathing from the AeroTech II jet nebulizer.

FIG. 10 is a plot of upper airway deposition as a percentage of total deposition plotted against body surface area from a group of patients inhaling pentamidine aerosols from AeroTech II type nebulizers.

FIG. 11 (right side) shows particle distributions for I-neb membranes (blue-study 1, red-study 2, dotted line AeroTech II reference). Two images are shown (left side) with corresponding aerosol distributions.

FIG. 12 shows one embodiment of a holding chamber designed to improve delivery of vibrating membrane aerosols by connecting to the aerosol generator. One approach to directing expiration is shown.

FIG. 13 shows another embodiment of a holding chamber designed to improve delivery of vibrating membrane aerosols by connecting to the aerosol generator. A different approach to directing expiration is shown.

FIG. 14 shows yet another embodiment of a holding chamber designed to improve delivery of vibrating membrane aerosols, where the vibrating membrane is in the floor of the chamber (there is no need for connecting to the aerosol generator through a conduit). One approach to directing expiration is shown.

FIG. 15 shows yet another embodiment of a holding chamber designed to improve delivery of vibrating membrane aerosols, where the vibrating membrane is in the floor of the chamber (there is no need for connecting to the aerosol generator through a conduit). A different approach to expiration is shown.

FIG. 16 shows pictures of commercially available vibrating mesh nebulizers, some of which can and should be used with the holding chamber described herein.

FIG. 17 shows bar graphs depicting Inhaled Mass (IM) presented as a percent of the initial nebulizer charge. ‘COPD’ breathing pattern (Vt 450 mL, rate 15, duty cycle 0.35, panel A), “Slow and Deep” pattern (Vt 1500 mL, rate 5, duty cycle 0.70, panel B).

FIG. 18 plots Standing Cloud particle distributions for 3 Omron nebulizers (with the AeroTech II jet nebulizer results [dotted line] provided as a reference).

FIG. 19 plots particle distributions measured during ventilation for the 3 Omron nebulizers, but without the holding chamber of the present invention.

FIG. 20 plots particle distributions measured during ventilation for the 3 Omron nebulizers, but with the holding chamber added.

FIG. 21 plots ventilated particle distributions with the holding chamber using COPD breathing pattern at different RH; 21%, open circles [MMAD=1.26], 32% open squares [MMAD=1.18], 50% closed circles (not seen as superimposed on open circles) [MMAD=1.27] and closed rectangles at 90% RH [MMAD=1.45].

FIG. 22 provides both particle distributions (Panel C) and scintigraphy images (Panels A and B) from a volunteer using the holding chamber. The subject inhaled radiolabeled particles from chamber circuit producing leftward distribution (closed circles), image A from modified Omron #1 no upper airway deposition seen; Image B same subject inhaling from chamber and Omron #3 circuit producing rightward distribution (open circles), upper airway deposition measured to be 13%.

FIG. 23 is a scintigraphic image (anterior view) from normal volunteer following inhalation of radiolabeled amikacin aerosol using Aeroneb nebulizer and Idehaler.

FIG. 24 shows pictures of the Idehaler (La Diffusion) and a holding chamber of the present invention, and compares features.

FIGS. 25A-C show the results where the Idehaler from La Diffusion was tested in the in vitro bench setup against a holding chamber of the present invention using the Aeroneb nebulizer.

FIG. 26A shows an experimental setup using a chamber with a jet nebulizer. FIG. 26B shows an experimental setup using the jet nebulizer without a chamber.

FIG. 27A is a plot showing the results (using the experimental setups shown in FIGS. 26A and 26B) with and without the chamber for an AeroEclipse jet nebulizer using the “COPD” breathing pattern (tidal volume 450 ml). FIG. 27B is a plot showing the results (using the experimental setups shown in FIGS. 26A and 26B) with and without the chamber for an AeroEclipse jet nebulizer using the “Slow and Deep” breathing pattern (tidal volume 1.5 liters).

FIGS. 28A and B are bar graphs depicting Inhaled Mass (IM) presented as a percent of the initial nebulizer charge. FIG. 28A shows the results (using the experimental setups shown in FIGS. 26A and 26B) with and without the chamber for an AeroEclipse jet nebulizer using the “COPD” breathing pattern (tidal volume 450 ml). FIG. 28B shows the results (using the experimental setups shown in FIGS. 26A and 26B) with and without the chamber for an AeroEclipse jet nebulizer using the “Slow and Deep” breathing pattern (tidal volume 1.5 liters).

FIG. 29 shows the “standing cloud” results for a commercial nebulizer utilizing 3 different membranes (represented by A, B and C). The circles and squares show the results for two runs for each membrane.

FIG. 30 shows an experimental setup using a chamber with a narrowing tube or stenosis in the context of a commercial nebulizer. While FIG. 30 shows a tube with a T shape, it need not be a T at all—but could just be a straight narrow tube with an obstruction.

FIG. 31A is a plot showing the results (using the experimental setup shown in FIG. 29—with and without the narrowing tube or stenosis) for an Aeroneb Solo nebulizer using the “COPD” breathing pattern (tidal volume 450 ml). FIG. 31B is a plot showing the results (using the experimental setup shown in FIG. 29—with and without the narrowing tube or stenosis) for an Aeroneb Solo nebulizer using the “Slow and Deep” breathing pattern (tidal volume 1.5 liters).

FIGS. 32A and B are bar graphs depicting Inhaled Mass (IM) presented as a percent of the initial nebulizer charge. FIG. 32A shows the results (using the experimental setup shown in FIG. 29—with and without the narrowing tube or stenosis) for an Aeroneb Solo nebulizer using the “COPD” breathing pattern (tidal volume 450 ml). FIG. 32B shows the results (using the experimental setup shown in FIG. 29—with and without the narrowing tube or stenosis) for an Aeroneb Solo jet nebulizer using the “Slow and Deep” breathing pattern (tidal volume 1.5 liters).

FIG. 33 shows one embodiment of a narrowing tube or stenosis contemplated by the present invention. FIG. 33A is a side-view photograph of the “Beige-T” stenosis. FIG. 33B is an inside-view photograph of the “Beige-T” stenosis showing the obstruction or baffle. FIG. 33C is a side-view sketch of the “Beige-T” stenosis. FIG. 33D is an inside-view sketch of the “Beige-T” stenosis showing the obstruction or baffle extending into the middle of the inner diameter to a distance of approximately (plus or minus 10%) the radius of the inner diameter.

### DETAILED DESCRIPTION

The deposition of inhaled drugs in the lungs is affected by many factors, particularly the efficiency of the device, the size of the generated particles, mixing of the aerosol with room air, the breathing pattern and the inter-device variability of the nebulizer itself. In one embodiment, the present invention contemplates a chamber that mitigates many of these issues and allows control of the inhaled mass of a drug, the need for breath actuation, the breathing pattern (which affects both inhaled mass and deposition in the lungs) and particle distribution (removing large particles that deposit in the throat) without sophisticated electronics. Thus, aerosolized drug delivery with the presently described VHC device combined with a vibrating membrane nebulizer is independent of breathing pattern, does not require breath actuation and does not require sophisticated technology to control breathing.

We have studied these devices and published summaries of their function (J Aerosol Med Pulm Drug Deliv. 2012; 25(2):79-87; J Aerosol Med Pulm Drug Deliv. 2010; J Aerosol Med. 1998; 11 Suppl 1:S105-111) and we have significant unpublished data illustrating these problems. In brief the current state of the art addresses only item number 3 above (the I-neb) a smart nebulizer that measures patient breathing and trains the patient to breathe appropriately. This system is expensive and requires patient cooperation. In addition the I-neb does not address problems 1, 2 and to some extent 4.

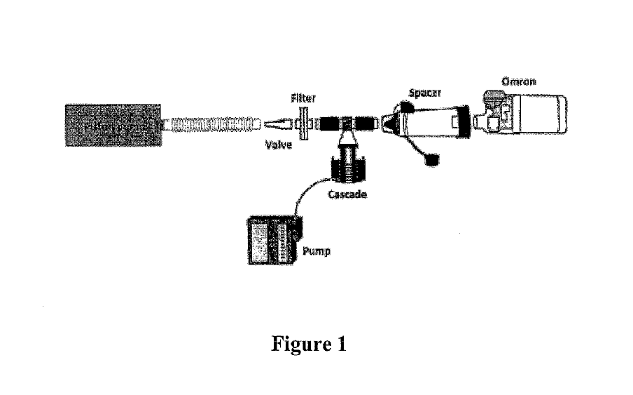

FIG. 1 combines two existing technologies to solve all of these problems. It comprises: 1) a vibrating nebulizer (e.g. Omron u22—pictured, the Aeroneb Go, the, the Mini-mist) (I-neb technology is NOT required) with 0.5 ml normal saline solution added to represent the drug (labeled with 99mTc), 2) a valved holding chamber with antistatic properties e.g. the InspiRx InspiraChamber., a low resistance flap valve, 3) a piston ventilator that mimics a patient breathing with different breathing patterns, 4) a cascade impactor to measure particle distribution, 5) a filter to capture particles that are not captured by the cascade and 6) an aerosol that consists of radiolabelled saline droplets. This setup therefore measures the effects of breathing pattern on nebulizer output, particle distribution, and inhaled mass.

In brief, the nebulizer is turned on and allowed to run either continuously or is manually turned on and off using its pushbutton switch (breath actuated). Aerosol enters the chamber and passes into the impactor or the filter, during expiration the exhaled gases pass out of the system via the low resistance flap valve. The inspiratory air stream can be modified by sealing the omron opening and allowing inspiratory gases to enter only via the inspiratory port on the VHC (not shown).

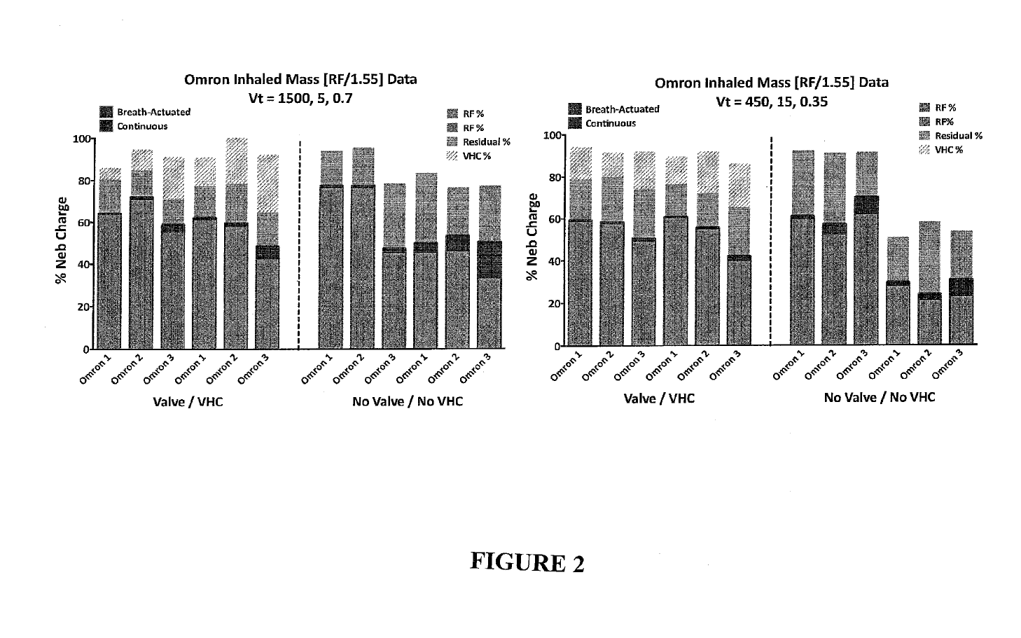

Data shown in FIG. 2 represents all of the radioactivity added to the nebulizer (a mass balance). Data are shown for 3 devices. The left represents slow and deep breathing, with a rapid expiration; while the right represents a more rapid pattern consistent with a patient with COPD (low inspiratory volume prolonged expiration higher breathing frequency. The goal is a device that is easy to use (no push button actuation), inexpensive (no smart technology), efficient, providing delivery independent of breathing pattern with no large particles in the distribution. In FIG. 2, the blue bars represent manual breath actuation, the red bars continuous operation, dark grey bars are activity remaining in the nebulizer, light grey bars activity in the VHC, each colored bar represents the inhaled mass (cascade impactor activity plus the filter activity), the lighter color bars are the respirable mass (the latter is the aerosol distribution with all particles 1.5 microns and above excluded). Differences in total activity from 100% represents aerosol lost to the environment.

FIG. 2 demonstrates the following: 1) continuous operation using the VHC device results in aerosol delivery very close to breath actuation (e.g. breath actuation is not needed), 2) using the VHC device results in delivery independent of breathing pattern (e.g. the red bars for both breathing patterns are very similar using the VHC device and they drop by 50% with no device for the smaller tidal volume and 3) large particles are minimized with the VHC device as shown by the reduction in the dark colored bars with VHC

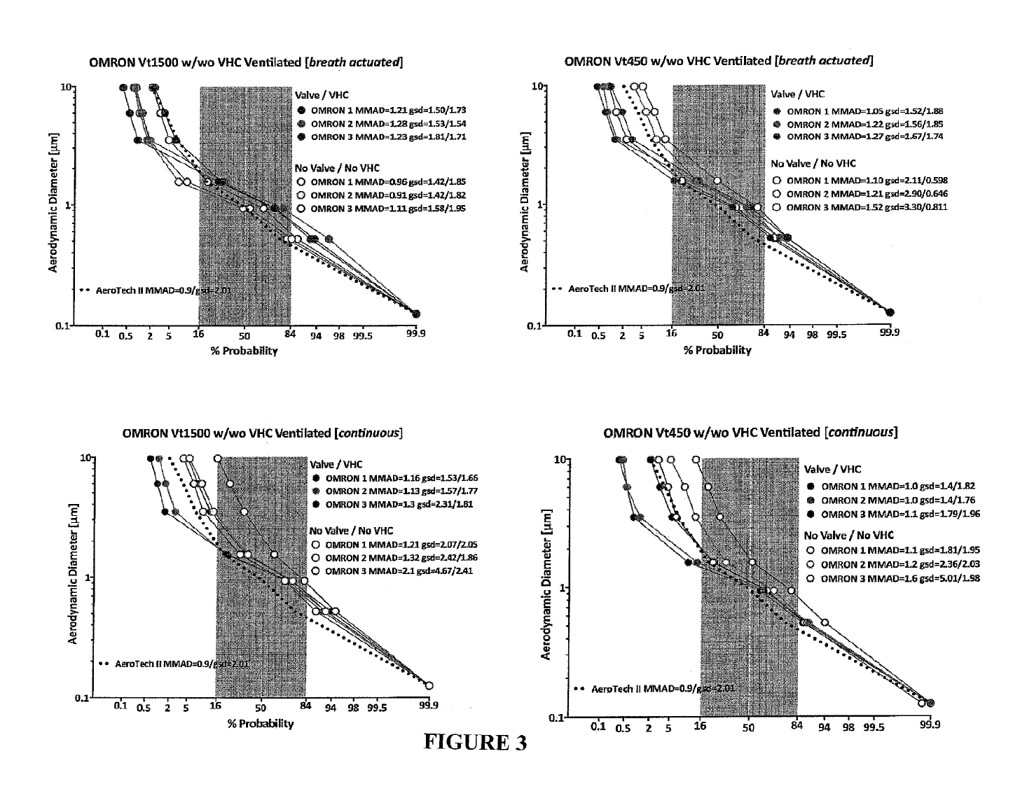

Actual particle distributions are shown in FIG. 3. The slow and deep pattern data is on the left and the COPD pattern on the right. Open circles are distributions without the VHC device, filled circles are with the VHC. The distributions are determined by three factors: 1) the individual device, e.g. #3 makes bigger particles than the others, 2) the presence of the VHC-all distributions with the VHC have significantly fewer particles above 1.5 microns and 3) the use of breath actuation-distributions with breath actuation are better than continuous ventilation but only if the VHC device is not used. With the VHC, distributions are optimal with or without breath actuation. The dotted line represents the best jet nebulizer tested in our lab, the AeroTech II. That device results in upper airway deposition in adults of less than 5% (J Aerosol Med 1998; 11 Suppl 1:S105-111) As shown above, the addition of the VHC provides equal or better distributions (fewer large particles) during continuous ventilation as during the more complex maneuver of breath actuation.

Variation between Omron devices is inherent in their performance. It has been previously shown by our group that variation in output and particle sizes is due to inconsistencies in the vibrating membrane between devices (J Aerosol Med Pulm Drug Deliv. 2010) but the addition of the VHC device reduces this variability by taking out the largest particles. Finally it is important to note that by varying the opening in the VHC, inspiratory flow can be regulated without electronics. If slow and deep breathing is desired the VHC has an orifice that limits flow and gives audible feedback to the patient.

A. Ensuring Effective Lung Delivery with Vibrating Mesh Nebulizers

The disadvantages of wet nebulizers are well known. While they allow flexibility in drug delivery, they require compressed gas, they are inefficient and they generate polydisperse aerosols. A modern solution is the vibrating mesh nebulizer. Powered by electricity, the vibrating mesh does not require compressed gas and is capable of high efficiency. However, from a practical point of view, an efficient vibrating mesh system can be just as inefficient as a typical jet nebulizer. In addition, the particles from vibrating systems can be even more polydisperse and variable from mesh to mesh than aerosols from jet nebulizers. Some of these problems can be addressed by electronic control systems, for example, breath-actuation. Unfortunately the use of breath-actuation significantly lengthens treatment time. Further, while breath-actuation can avoid expiratory losses, simple breath actuation does not control the pattern of breathing which is also important in drug delivery. The latter problem has been addressed by more sophisticated control systems such as those used by Akita (ActivAero, Wohra Germany) and I-neb (Philips Respironics, Parsippany N.J.).

Work presented herein outlines the problems and differences in delivery between jet and mesh nebulizers from an experimental point of view as demonstrated by bench testing. We relate the bench model used in our laboratory to actual delivery of wet aerosols to the lungs of humans and introduce a new device designed to address many of the problems described above that does not have sophisticated control systems. The goal of the new device is to deliver wet aerosols efficiently to humans with minimal losses, independently of breathing pattern with a reduced treatment time. In addition, the device should minimize the polydispersity of the aerosols produced by the mesh to avoid deposition in the upper airways.

B. Principles and Approach

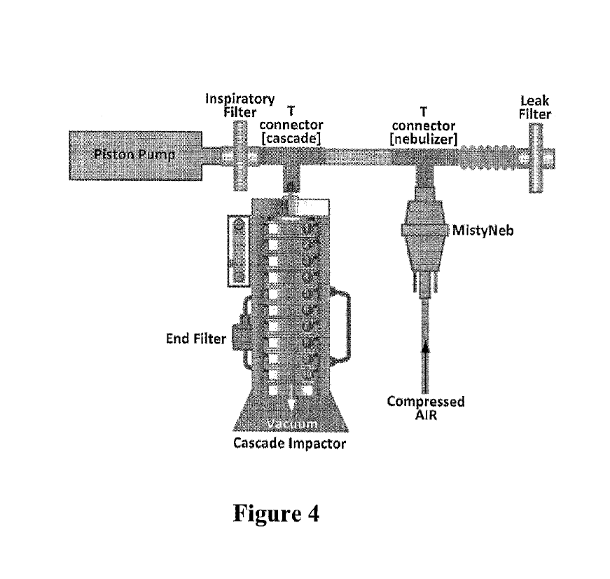

We believe that wet aerosols should be measured under conditions of actual use. Particles that enter the patient's respiratory tract mix with room air, which affects the aerosol by partial evaporation before the particles are inhaled. Therefore, we test aerosol systems using breathing patterns that are reasonable facsimiles of actual patient patterns, e.g. adult vs child, COPD vs normal (J Aerosol Med Pulm Drug Deliv. 2009; 22 (1):11-18; J Aerosol Med Pulm Drug Deliv. 2009; 22(1):9-10; J Aerosol Med. 1991; 4(3):229-235). For example in FIG. 4 we test a jet nebulizer. Aerosols are drawn into the cascade impactor and the inspiratory filter by a piston pump that generates various breathing patterns. As shown in the figure a typical jet nebulizer the “Misty Neb” (Allegiance, McGraw Park, Ill.) is in line with a low flow cascade impactor and a filter placed to capture “inhaled particles”. Aerosols captured in the cascade and the filter comprise the “inhaled mass” or all the drug that would be inhaled by a patient breathing in a manner duplicated by the piston pump. We have tested many nebulizers using this technique including vibrating mesh devices.

FIG. 4 depicts a bench setup for determining inhaled mass and particle distribution for a jet nebulizer for both ‘standing cloud’ (no ventilation) and during breathing. The piston pump is designed to mimic various breathing patterns used by patients (J Aerosol Med. 2003; 16(4):379-386).

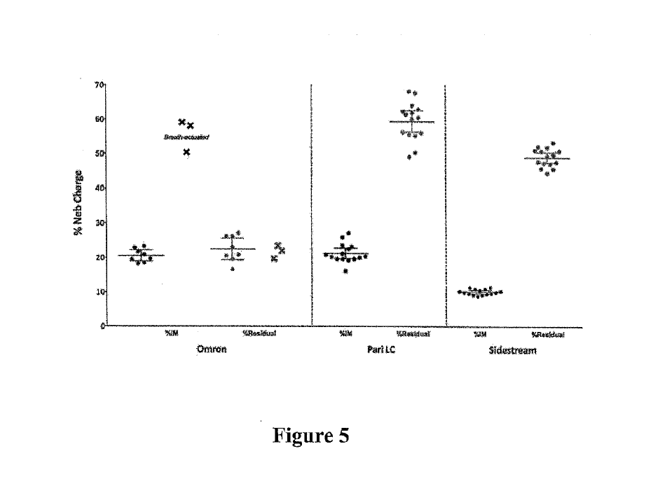

Typical results are shown in FIG. 5. Inhaled mass and nebulizer residuals are shown as percentages of the nebulizer charge. The pattern of breathing used in these experiments was a “COPD” pattern (tidal volume 450 Ml, resp rate 15/min and duty cycle of 0.35, modified from ref 5, X's depict effects of breath-actuation). The Omron U22 (Omron Healthcare Inc., Bannockburn, Ill.) is compared to two commonly used jet devices, the Pari LC jet plus (Pari Respiratory Equipment, Midlothian, Va.) and the Respironics Sidestream (Philips Respironics, Parsippany, N.J.) (J Aerosol Med Pulm Drug Deliv. 2010). The important observations are that the inhaled mass of the mesh device is no better than that of the jet devices in spite of the fact that the residuals of the jet nebulizers are much higher than the Omron. Obviously the Omron's aerosol is lost during expiration. The addition of “breath-actuation” illustrates this phenomenon. On FIG. 5 the “breath-actuation” data points for the Omron were obtained by manually triggering the devices on/off button in sync with the inspiratory phase of the piston ventilator. While this maneuver is obviously cumbersome, inhaled mass increases to over 50%.

C. Particle Distributions and Lung Deposition Using Jet Nebulizers

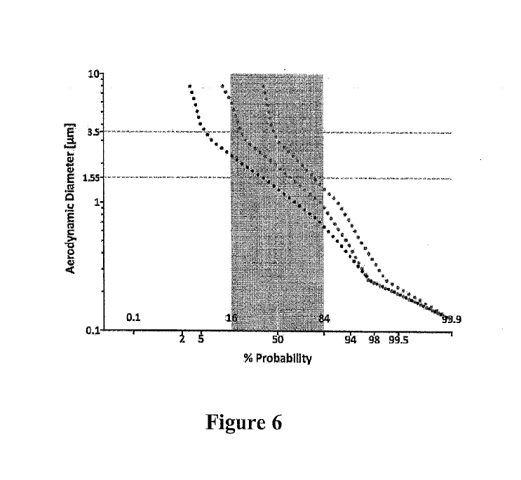

Aerosols from different wet nebulizers sampled by cascade impaction and plotted on log probability paper are illustrated in FIG. 6. Average distributions from previous studies for jet nebulizers; green dots Misty-Neb, red dots AeroEclipse, black dots AeroTech II. Average distributions from three common devices are shown; the Misty-Neb, AeroEclipse and AeroTech II (Biodex Medical Systems, Shirley, N.Y.) (J Aerosol Med. 2003; 16(4):379-386; J Aerosol Med Pulm Drug Deliv. 2010; J Aerosol Med. 1988; 1:113-126). This figure demonstrates that different nebulizers can produce different aerosol distributions. They appear multi-modal with varying amounts of “large” particles (e.g. on the left of each distribution). Over the years we have correlated these distributions with deposition scans in patients. This paper focuses on the partitioning of deposited particles between the lung parenchyma and upper airways.

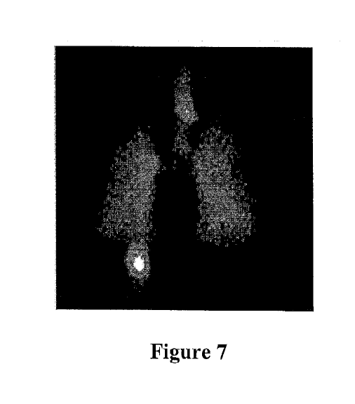

FIG. 7 illustrates the deposition image of a patient following inhalation during tidal breathing from the Misty-Neb (J Aerosol Med. 2003; 16(4):379-386). Following a drink of water, upper airwayactivity (mouth, throat) was washed into the stomach and easily scanned and quantified. For this subject 68% of the deposited particles were found in the stomach.

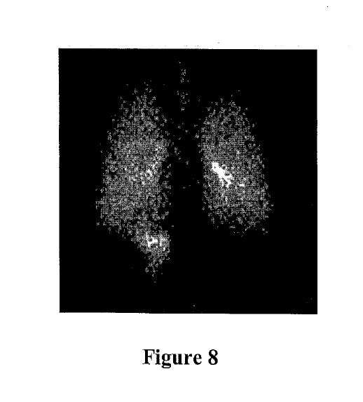

FIG. 8 represents another image from the same patient of deposition following inhalation of interferon aerosol from the AeroEclipse. Compared to FIG. 7, there is a clear shift of deposition with an increased fraction in the lungs (only 28% in the stomach). The changes in the images reflect the changes in the aerosol distributions shown in FIG. 6.

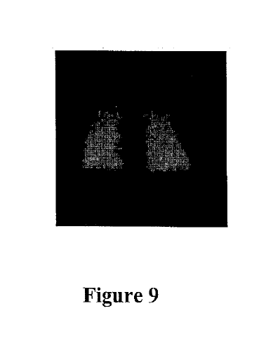

FIG. 9, is a Gamma camera image from another patient following inhalation of pentamidine aerosol during tidal breathing from the AeroTech II nebulizer (Am Rev Respir Dis. 1991; 143(4 Pt 1):727-737). We have used the AeroTech II in many human studies over the years and as shown in the Figure upper airway deposition for this device is minimal (no stomach activity) corresponding to the most leftward aerosol distribution shown in FIG. 6.

In general, in our hands, we find that, in adults, wet aerosol particles inhaled during tidal breathing will bypass the upper airways if they are less than about 2.5 μm in diameter when measured by the technique shown in FIG. 4 (J Aerosol Med. 2003; 16(4):379-386).

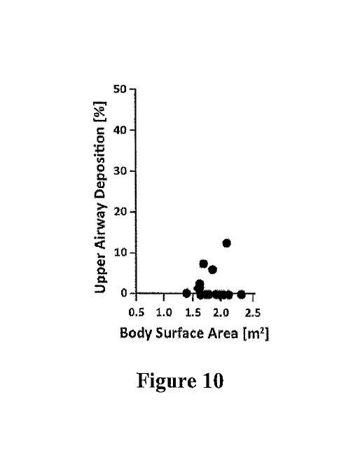

Consistent with that statement, the AeroTech II distribution sets our “standard” in that we generally see 5% or less upper airway deposition for this device (FIG. 10) (J Aerosol Med. 1998; 11 Suppl 1:S105-111). Upper airway deposition as a percentage of total deposition plotted against body surface area from a group of patients inhaling pentamidine aerosols from AeroTech II type nebulizers. Many values are superimposed near 0%⁸.

D. Vibrating Membrane Devices and Deposition

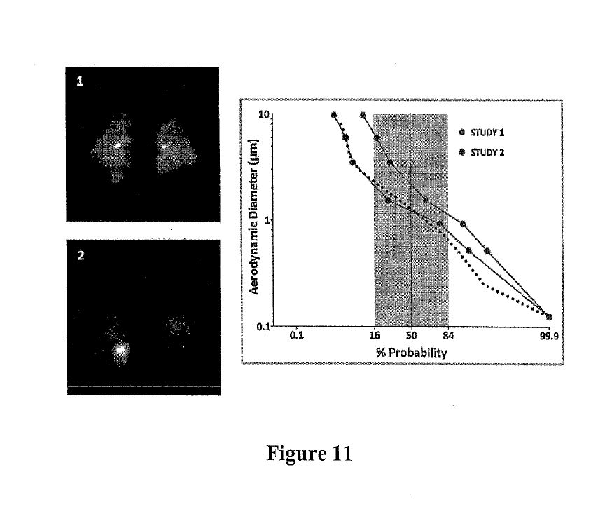

In 2011 we published our experience using the relatively sophisticated I-neb to deliver interferon aerosols to patients with IPF (J Aerosol Med Puk Drug Deliv. 2012; 25(2):79-87). In that study, we performed serial deposition studies in patients over 6 months. A typical example is shown in FIG. 11. Serial deposition studies in an IPF patient being treated with inhaled interferon aerosol (J Aerosol Med Pulm Drug Deliv. 2012; 25(2): 79-87). Particle distributions for the corresponding I-neb membranes are shown (blue-study 1, red-study 2, dotted line AeroTech II reference). Two images are shown with corresponding aerosol distributions. The upper panel demonstrates the lungs with little stomach activity.

The corresponding aerosol measured by our bench technique is illustrated on the panel to the right (filled blue circles). This distribution closely approximates that of a reference plot of the AeroTech II (dotted curve). On the next image is the second study performed with another I-neb device. There is significant stomach activity likely due to the fact that the membrane in this experiment produced larger particles (shown as filled red circles). With this shift in particle distribution, stomach activity increased from 5% to 30%.

E. Use of an Ultrasonic Chamber to Capture Aerosol

In summary, our data indicate that the more an aerosol distribution approaches that of the AeroTech II, fewer particles will deposit in upper airways. Vibrating mesh systems, like jet nebulizers, can be inefficient in lung delivery if 1) the aerosol they produce is lost during expiration 2) the mesh produces large particles and they are deposited in the upper airways and 3) the treatment times are long (necessitated by breath-actuation). To improve aerosol delivery, therefore, it would be desirable to capture more of the particles lost during expiration in a way that does not require breath-actuation and reduce the percentage of large particles before they are inhaled to prevent upper airway deposition.

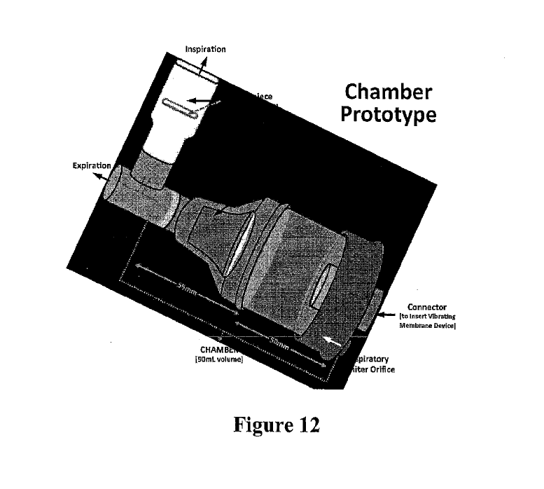

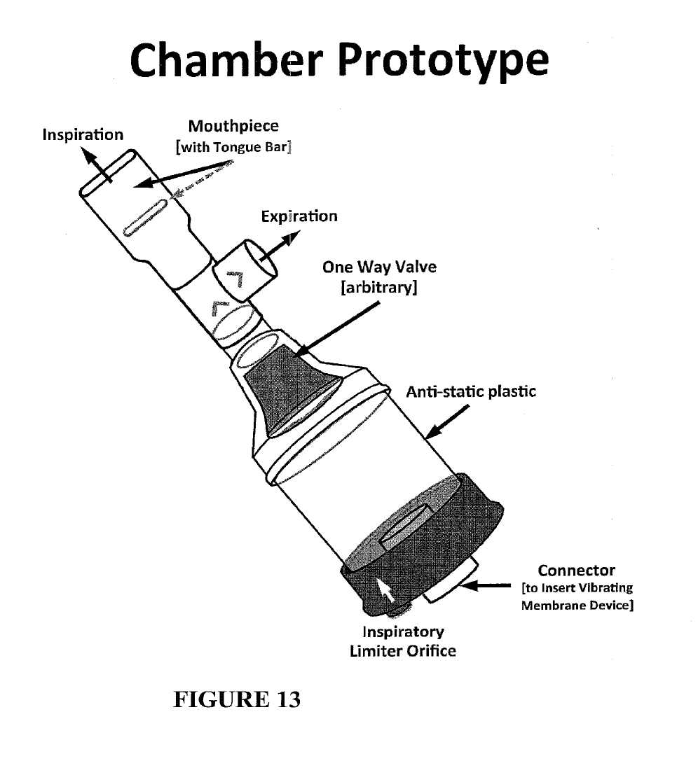

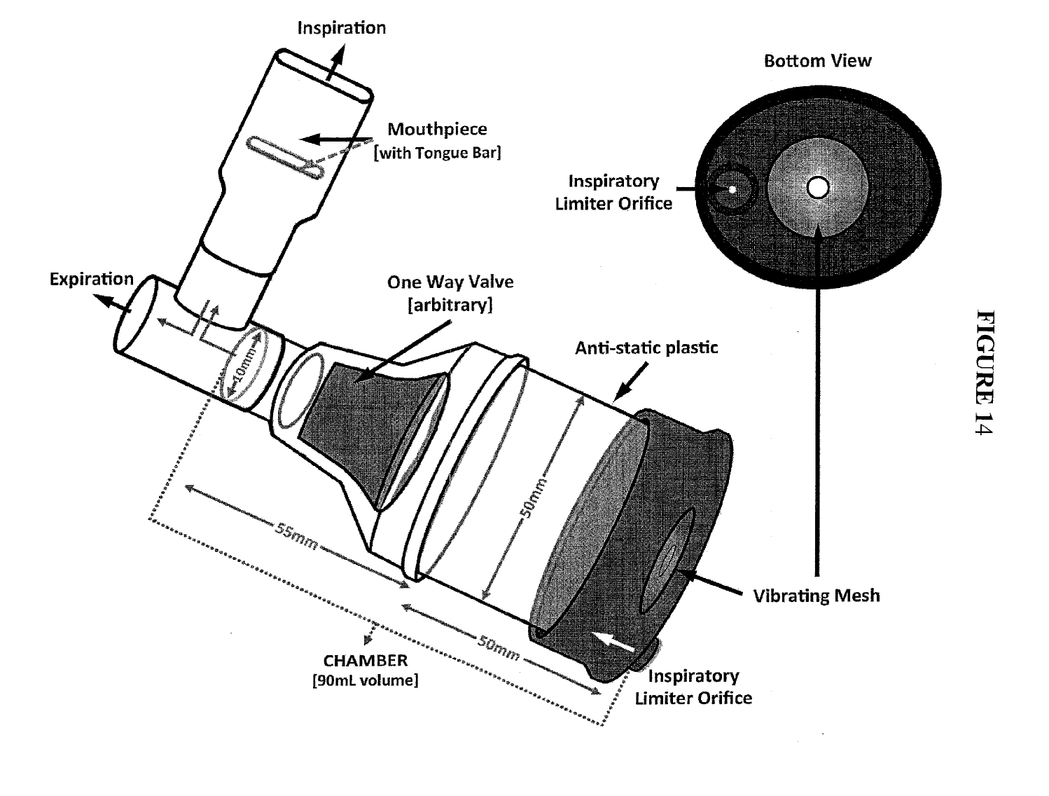

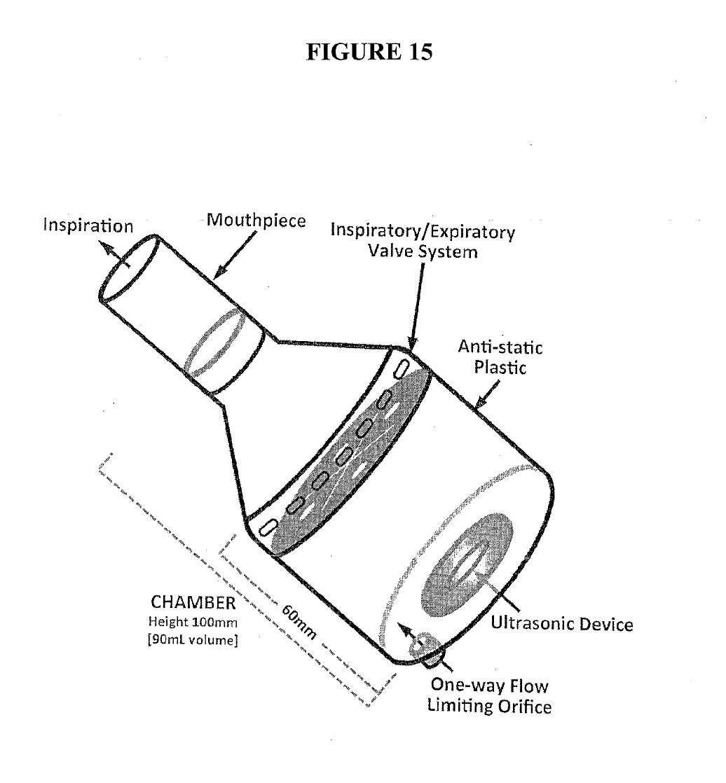

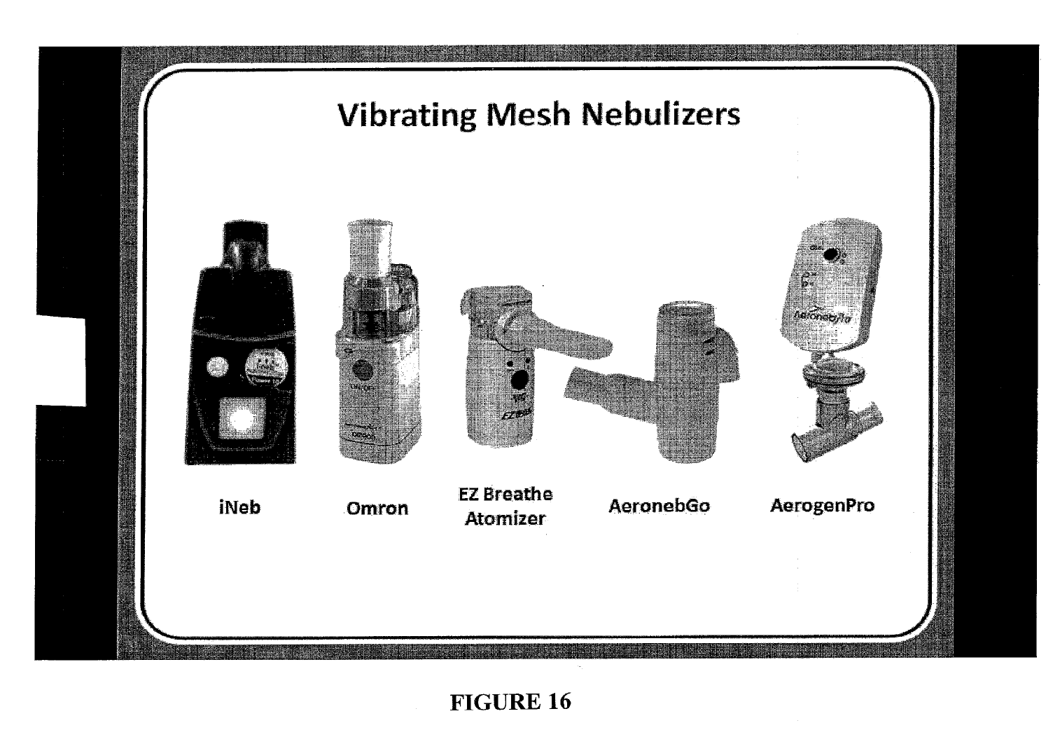

FIGS. 12 and 13 illustrate a chamber designed to improve delivery of vibrating membrane aerosols. This prototype chamber is designed for controlling aerosol delivery from ultrasonic membrane nebulizers. The tongue bar on the mouthpiece is a reference point for the patient to keep the tongue out of the way. When attached to the ultrasonic source, the chamber captures all emitted aerosol particles from a vibrating membrane system. The nebulizer runs continuously so breath actuation is not needed. The chamber captures all particles and holds them until the patient inhales. Inspiratory flow can be controlled via inspiratory resistances. During this time the aerosol is “conditioned”, that is there is partial evaporation and the larger particles, in particular, get smaller. One or more valves at the mouthpiece prevent backflow of gases during expiration.

Preliminary data indicate that for the U22 tested in FIG. 5, using the chamber, the inhaled mass during continuous operation increases from 20% to between 50-65%, the same as that of breath actuation. Breathing time is greatly reduced from that of breath-actuation (breathing times reduced by 50-75%, depending on the breathing pattern). Finally the chamber moderates the particle distributions with the primary effect on the larger particles. Repeated experiments yield aerosol distributions superimposed on the AeroTech II reference, markedly different from the distribution seen for the second study plotted in FIG. 11.

Below, this data is expanded and supported by human deposition studies, showing that the addition of the chamber to any vibrating system will provide maximal aerosol delivery to the lungs, bypassing the upper airways. Treatment time will be reduced without sophisticated electronic circuitry.

### EXPERIMENTAL

An important component of our in vitro testing technique is the use of low flow cascade impaction (≦2 L/min) to minimize effects of the impactor on nebulizer function (shown in FIG. 1). The probability plots (see FIG. 6) represent cascade impaction data from aerosols from different wet nebulizers. The intersection of the 2.5 μm line with a given distribution partitions the distribution between the ‘upper airways’ and the ‘lungs’. The stages above 2.5 μm ‘predict’ the percentage of upper airway deposition for that device. The rest of the distribution predicts deposition in the lungs. Supporting the in vitro predictions are representative images from human scintigraphy studies (FIGS. 7, 8 and 9). In our hands a measured aerosol distribution that is near to the distribution of the AeroTech II (Biodex Medical Systems, Shirley, N.Y.) jet nebulizer should have minimal upper airway deposition.

### Example 1

In this example, we tested 3 examples of the Omron U22 nebulizer. For each in vitro experiment, the nebulizer was filled (nebulizer charge) with 0.5 mL normal saline mixed with 400-900 μCi ^{99m}Technetium pertechnetate (^{99m} Tc). Radioactivity defining the nebulizer charge was measured in a dose calibrator (Biodex Medical Systems, Shirley, N.Y.). For each experiment, the nebulizer was run to dryness and the nebulizer reservoir measured for residual radioactivity.

We used a Harvard Pump (Harvard Apparatus, Millis, Mass.) to simulate two breathing extremes; the first with prolonged expiration, ‘COPD’ tidal volume of 450 mL, frequency of 15 breaths/min and duty cycle of 0.35, and the second, ‘Slow and Deep’, a pattern designed to maximize lung deposition, (tidal volume 1.5 liters, frequency 5 breaths/min and duty cycle of 0.70).

The chamber used was a modified valved holding chamber (VHC) (InspiraChamber, InspiRx Somerset, N.J., 170 mL), which is composed of antistatic plastic. As they pass through the chamber the particles are exposed to unsaturated room air, which enters the chamber through the inspiratory port of the VHC and the plastic nebulizer connector. Our laboratory has studied several configurations of this device with different chamber volumes and valve configurations. In this experiment, we report on the in vitro behavior of the 170 mL chamber.

To measure particle distribution we used a 7 stage Marple Cascade Impactor, with a 2.0 L/min vacuum flow (Thermo Fischer Scientific, Waltham, Mass.). Radioactivity from each stage was measured via calibrated ratemeter (Linak, Denmark).

Most of our experiments were carried out during months when relative humidity (RH) averaged 25%. To test the sensitivity of our experiments to changes in ambient humidity we placed our experimental apparatus in a tent containing a humidifier and repeated measurements for the COPD pattern at different RH. We were able to raise the ambient RH to 50 and 90%.

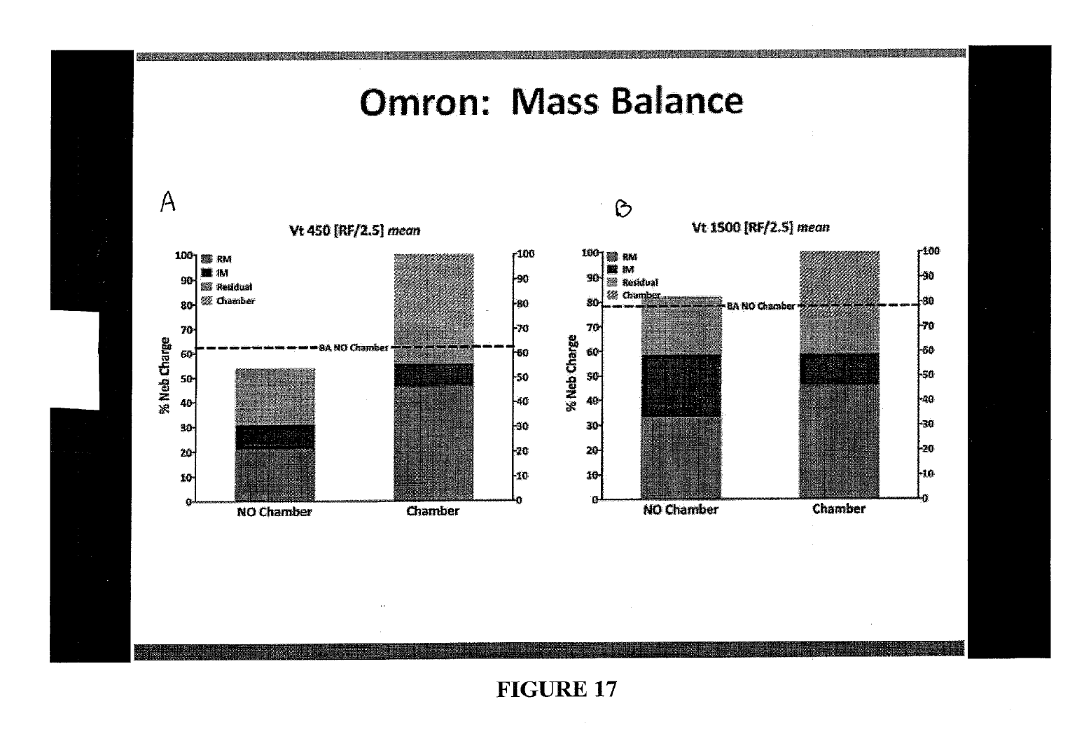

Average data from all these experiments are listed in Table 1. FIG. 17 shows the mass balance, expressed as a percent of the nebulizer charge. Mass balance measurements included IM, the device residual (losses were the exhaled fraction) and, for chamber experiments, chamber deposition. FIG. 17A represents the “COPD” breathing pattern and FIG. 17B represents the “Slow and Deep” breathing pattern. For the COPD pattern (no chamber) IM of 31.0±2.2% was similar to that first reported by Skaria et al. There was significant variation in IM with changes in breathing pattern with Slow and Deep IM=58.7±11.0. Breath actuation increased IM significantly for both patterns to 62.5±6.7% and 78.4±2.0 respectively. The increase in IM for the slow and deep pattern reflects the increase in duty cycle. Addition of the chamber reduced the variation in inhaled mass with breathing pattern. IM increased with both patterns of breathing to near that of breath actuation. With the chamber in place, we were able to account for nearly 100% of the initial nebulizer charge.

Nebulizer residuals ranged from 10-25% of the initial nebulizer charge with reduced residual when using the chamber suggesting that, during expiration, without the chamber, more particles impacted in the nebulizer as expiratory gases were exhaled into the nebulizer Chamber deposition, was about 25% of the nebulizer charge.

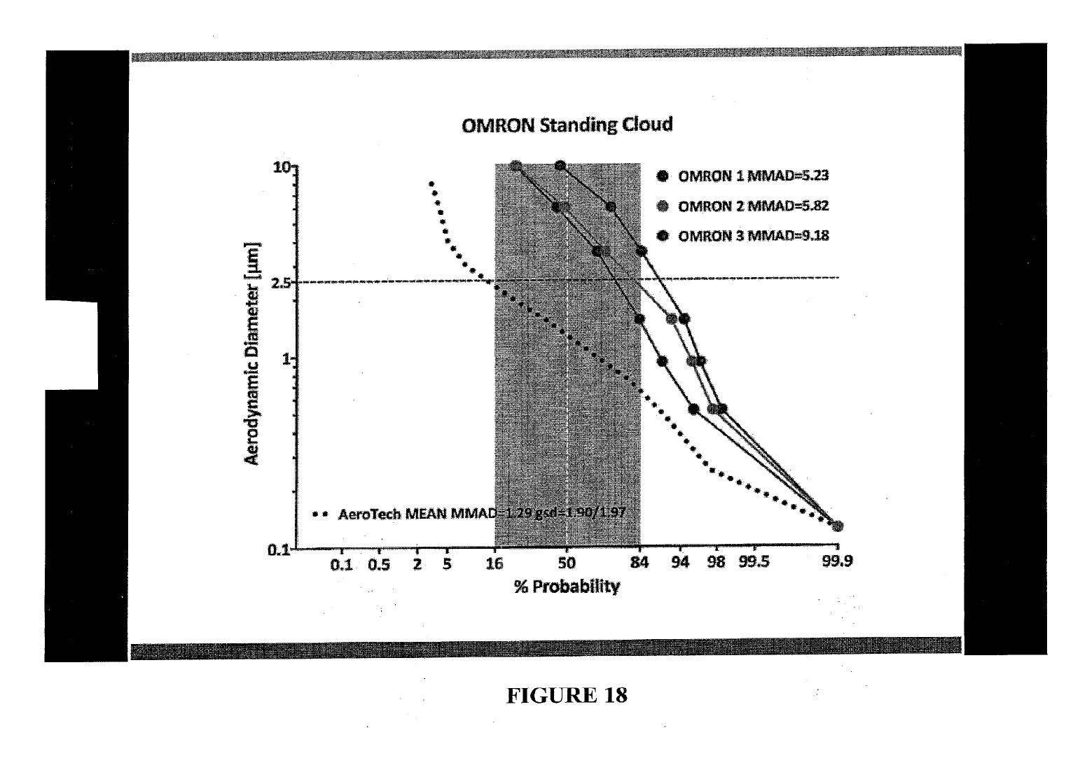

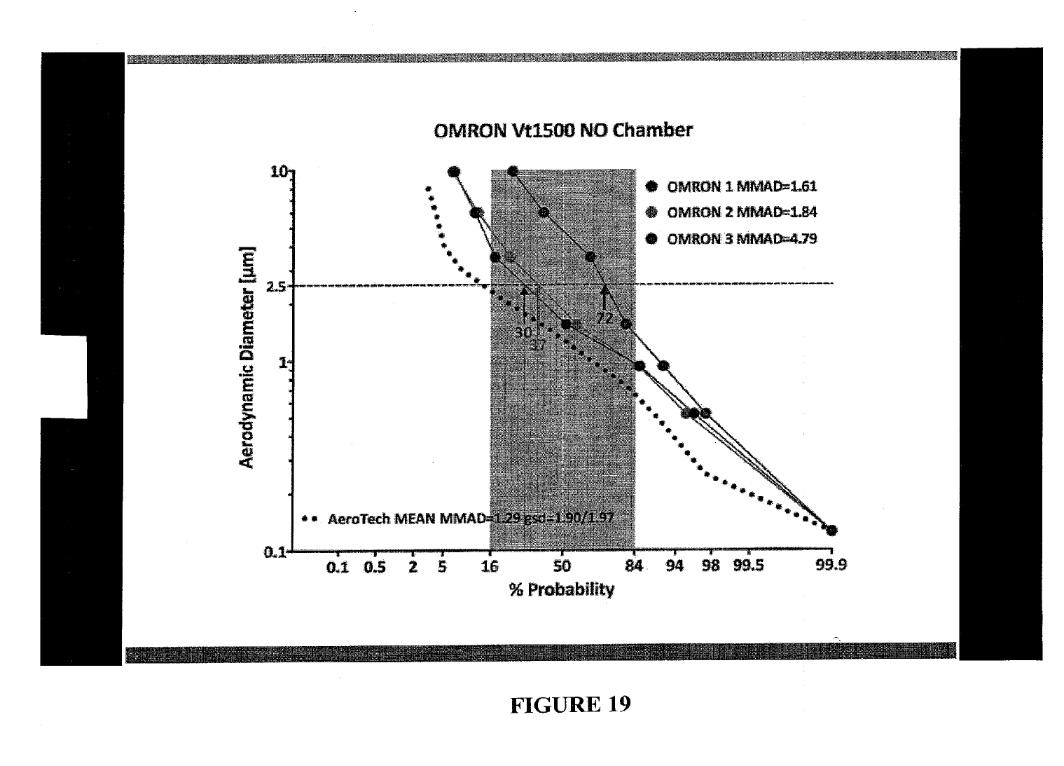

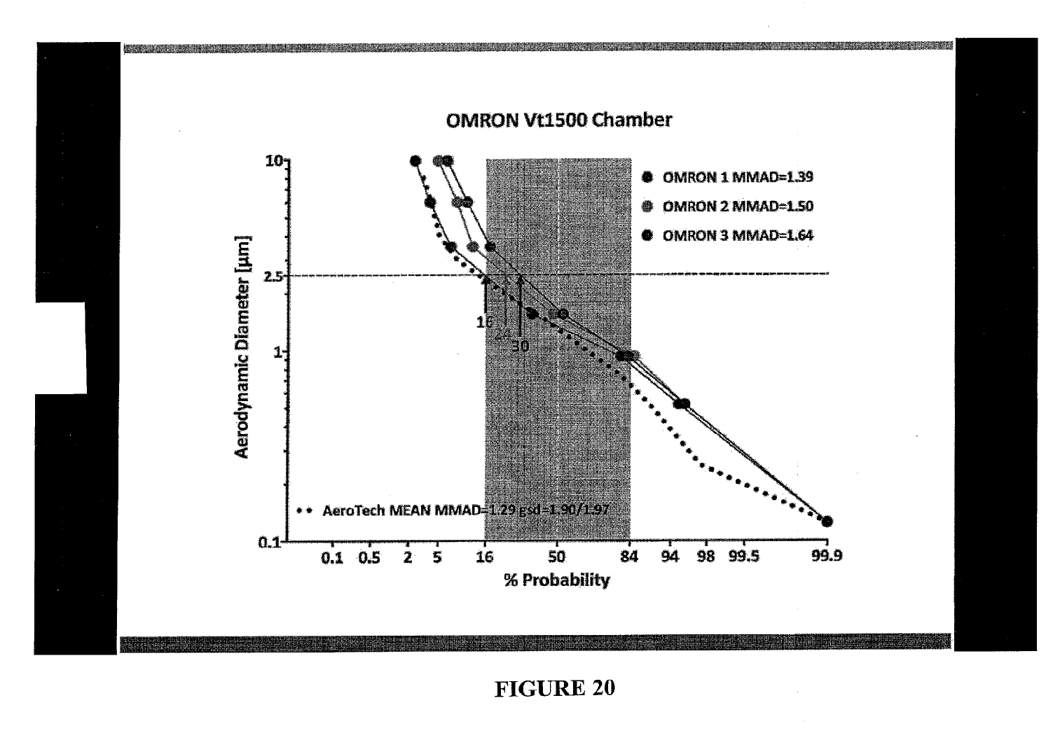

FIGS. 18, 19 and 20 depict the particle distributions for standing cloud, ventilated without chamber, and ventilated with chamber experiments. The data are superimposed on the AeroTech II composite (dotted line) for comparison. Values of RF are listed in Table 1. The standing cloud distributions indicate particles that are largely non-respirable with the average RF only 0.18±0.078. In addition, as we have found in previous studies, there is variation in distributions between devices. When ventilated (FIG. 19), each distribution shifts to the left, with an increase in the RF now ranging between 0.68±0.16 and 0.54±0.23. This effect is enhanced with the ventilated chamber (FIG. 20) with RF of 0.82±0.072 for the COPD pattern and 0.77±0.075 for Slow and Deep. More importantly the mean RF is affected by Omron #3, which produced significantly larger particles than the other devices. As shown in FIG. 20, for both patterns of breathing, the distributions of Omron #1 and 2 approximated that of the AeroTech II (dotted line).

The chamber influence on the respirable mass is shown on the mass balance plots in FIG. 17. The IM is partitioned into the RM by multiplying IM by the RF for each pattern of breathing, with and without the chamber. Without the chamber, between 10 and 30 percent of the Omron's output would be expected to deposit in the upper airways (up to 40% of the IM). With the chamber, two effects are seen; an increase in IM and RF with a significant increase in RM.

Mean treatment times are listed in Table 1. As indicated from the magnitude of the standard deviation, there is significant variability between devices. Continuous operation results in lower times for all patterns of breathing.

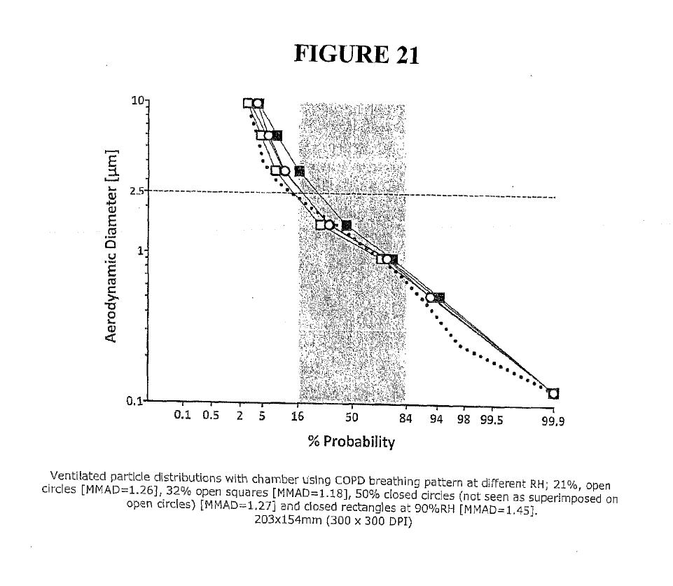

FIG. 21 and Table 1 summarize effects on particle distribution with large changes in RH. Changes in MMAD are small. There are small shifts in the particle distribution with mean RF ranging from 0.822 to 0.730 at the highest RH. These data suggest that humidity is not a significant factor in the final particle distribution leaving the chamber.

### Example 2

This example involves in vivo human studies. For this experiment, 150 μCi ^{99m}Tc was bound to sulphur colloid (^{99m}Tc-SC Pharmalucence, Inc., Bedford, Mass.). The purpose of these experiments was to further test the predictive value of our in vitro measurements on the regional distribution of deposition between the lungs and upper airways. Therefore we used two experimental conditions that produced distributions at the extremes of our testing e.g. relatively small and relatively large particles. Lung scintigraphy (Maxi Camera 400, General Electric, Horsholm, Denmark, Power Computing, Model 604/150/D, Austin, Tex., Nuclear MAC, Version 4.2.2, Scientific Imaging, Inc., CA)) was performed on a normal volunteer following inhalation of different aerosols (150 μCi ^{99m}Tc-SC) of nebulized saline, generated by different Omron devices using the chamber. Immediately after inhalation the subject swallowed a glass of water and the counts in the stomach used to estimate upper airway deposition (% total regional deposition). Data was compared with deposition achieved with the AeroTech II jet nebulizer (dotted line).

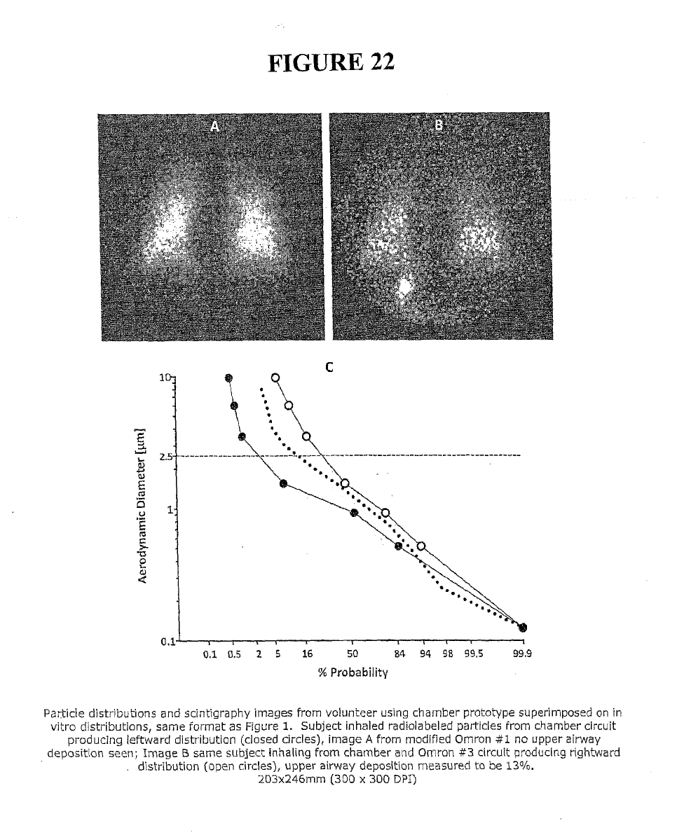

FIG. 22 illustrates lung deposition images in the same volunteer using the nebulizer with chamber system. The subject used a slow and deep pattern of breathing. The indicated particle distributions and corresponding images were measured following inhalation from a modified Omron #1 (image A) and Omron #3, the device with the lowest RF. The distribution to the left of the AeroTech II dotted line resulted in 100% deposition in the lung parenchyma (0% upper airway) and the distribution to the right of the AeroTech II had some upper airway activity (shown in the stomach), which represented approximately 13% of the total regional deposition.

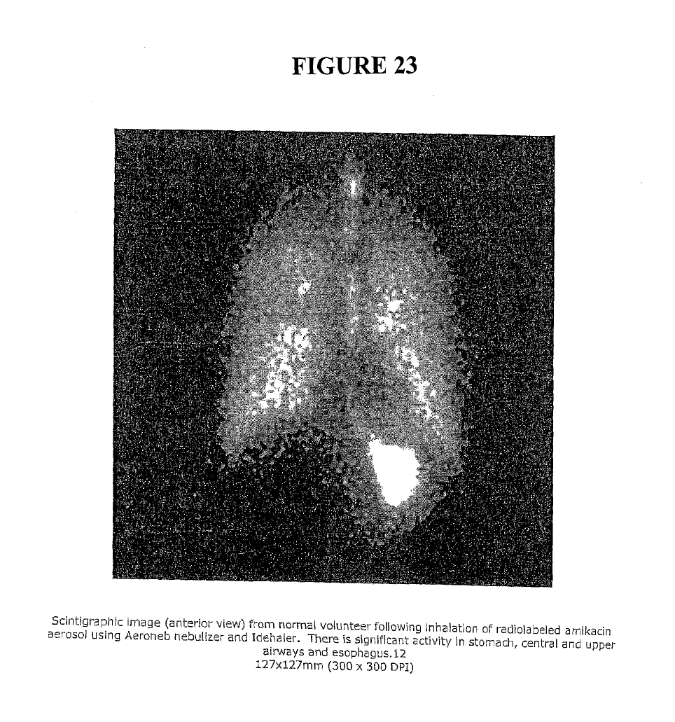

These results of low upper airway deposition contrast with the results of other investigators using other chambers. FIG. 23 is an image from a normal subject inhaling radiolabeled amikacin from the Idehaler (La Diffusion Technique Francaise, Saint Etienne, France). There is obvious marked stomach activity, central lung deposition, active mucociliary clearance and visible oropharyngeal activity, which in that study averaged 29.4±7.4% in 15 normal subjects. This pattern of delivery could pose a problem if the upper airway deposition resulted in local side effects.

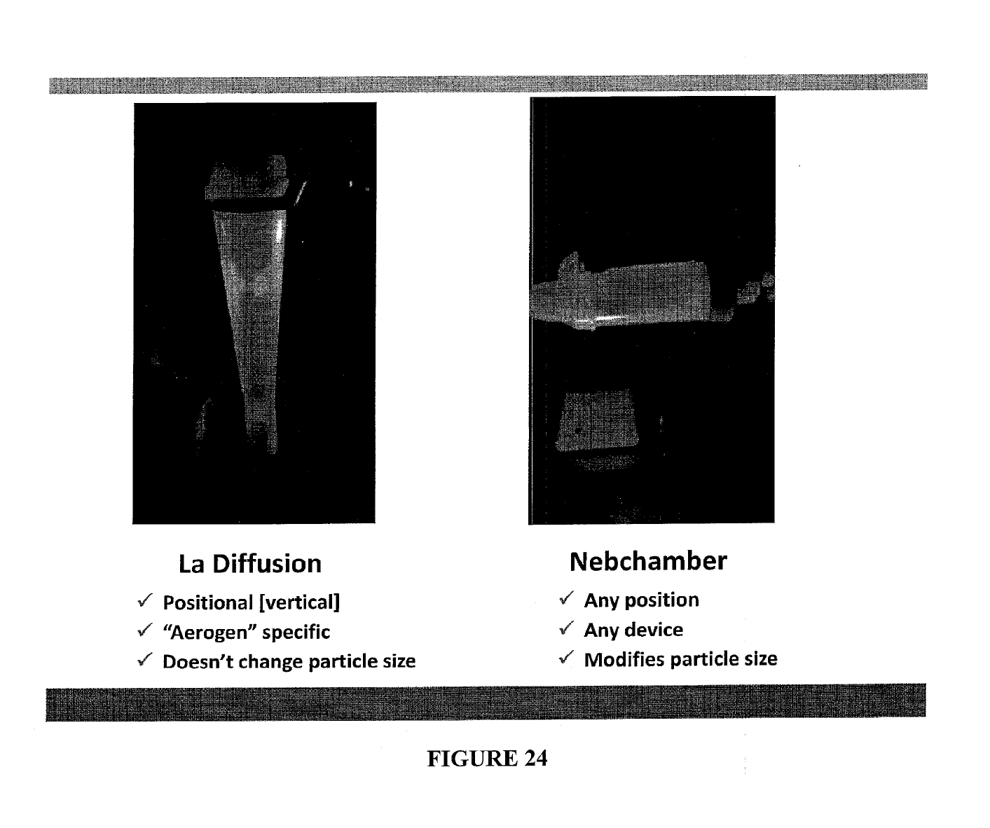

There are other disadvantages to the Idehaler from La Diffusion, including limits on how it is positioned and that it appears to be designed specifically for the Aerogen nebulizer. Most importantly, however, it doesn't change the particle size of the aerosolized droplets (perhaps because of the tapered design to accommodate the Aerogen plume) (FIG. 24).

### Example 3

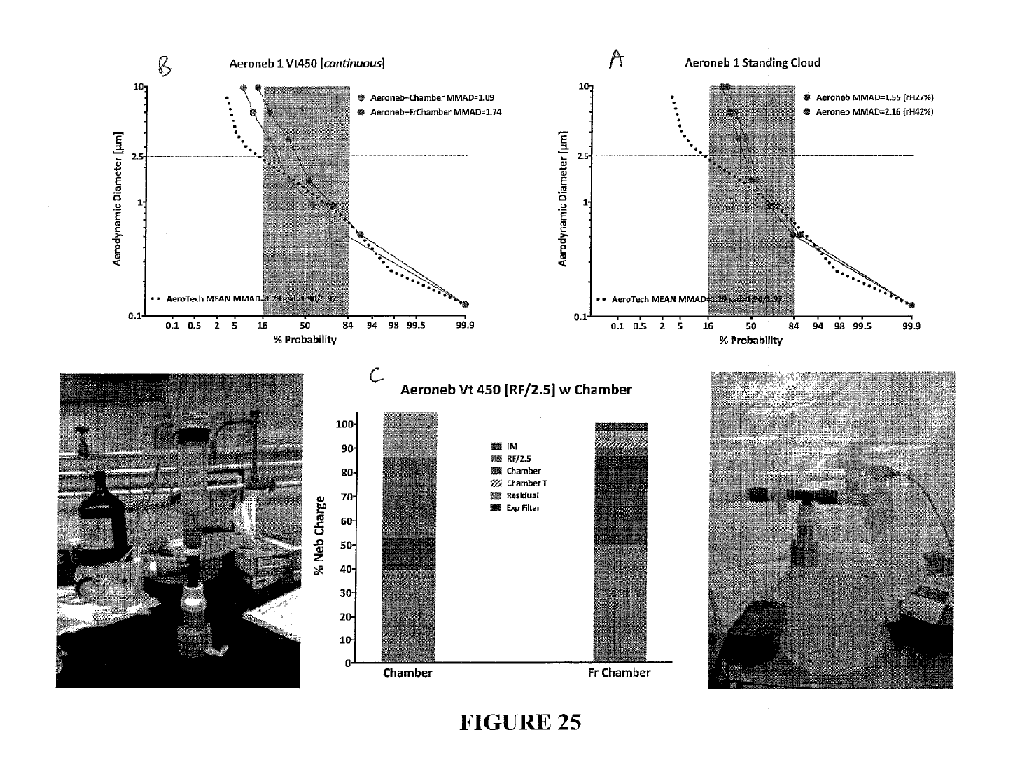

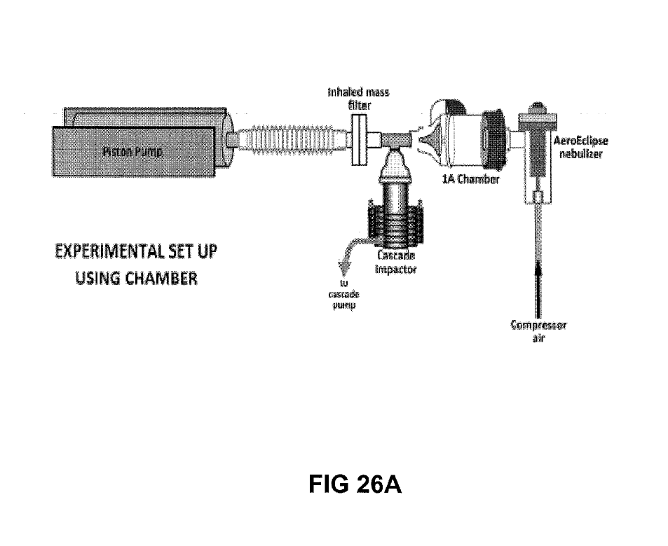

In this example, the Idehaler from La Diffusion was tested in the in vitro bench setup (as discussed in Example 1) against a holding chamber of the present invention using the Aeroneb nebulizer. The results are shown in FIGS. 25A-C.

FIG. 25A (upper right) shows the results for standing cloud aerosol distributions for the Aeroneb device with no chamber attached. Two runs were performed at different relative humidities (27% and 42%). The results show that approximately 50% of the particles will not enter the lungs and deposit in upper airway.

FIG. 25B (upper left) shows particle distributions during ventilation though the Idehaler (“Fr-chamber”) and holding chamber of the present invention. The results show that the holding chamber of the present invention shifted the particle size distribution to the left, indicating smaller particles.

Mass balance data (FIG. 25C) reveals that the French Idehaler chamber delivered 86% of the drug, but approximately 47% of the particles will not enter the lungs (no improvement over standing cloud) and deposit in the upper airways (right bar graph). The holding chamber (left bar graph) delivers a much better aerosol with similar lung delivery and a marked reduction in upper airway fraction because those particles deposited in the chamber rather than the upper airways. Said another way, the holding chamber of the present invention takes out particles that would otherwise be deposited in the upper airways.

### Example 4

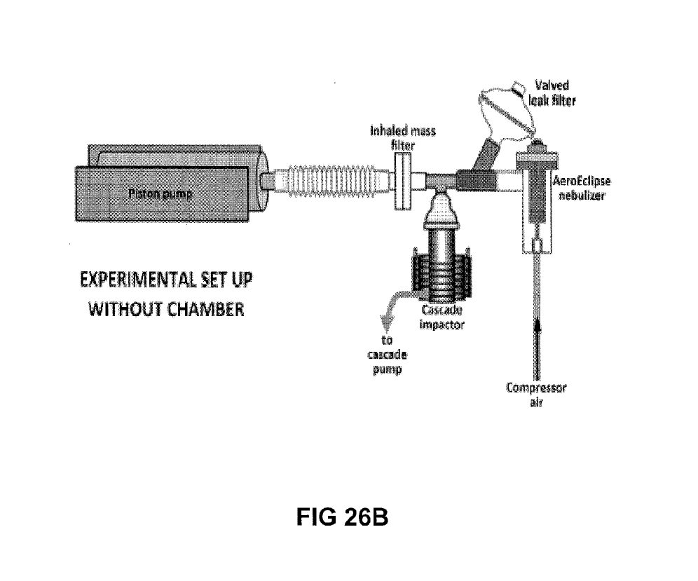

While the emphasis has been the use of the chamber for vibrating mesh devices, this example shows the general benefit of the chamber with aerosols, including those made by jet nebulizers. FIGS. 26A and 26B show the experimental setup with and without the chamber for an AeroEclipse jet nebulizer. This particular nebulizer can be run breath actuated or continuously. A pump was used to simulate two breathing extremes; the first with prolonged expiration, ‘COPD’ tidal volume of 450 mL, frequency of 15 breaths/min and duty cycle of 0.35, and the second, ‘Slow and Deep’, a pattern designed to maximize lung deposition, (tidal volume 1.5 liters, frequency 5 breaths/min and duty cycle of 0.70).

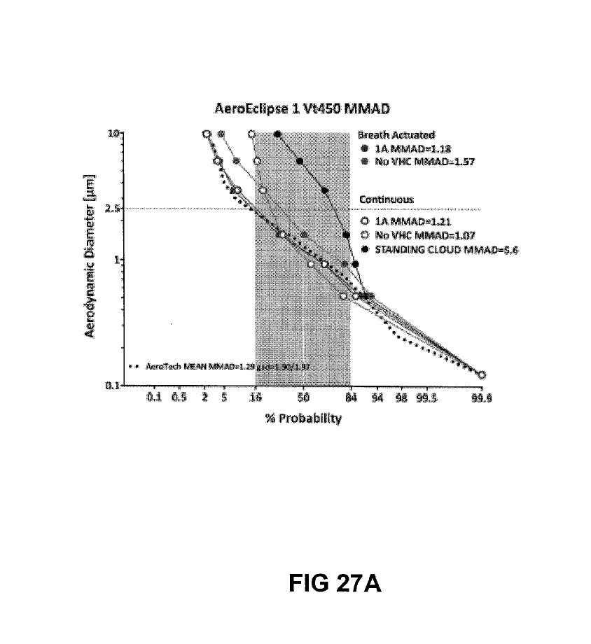

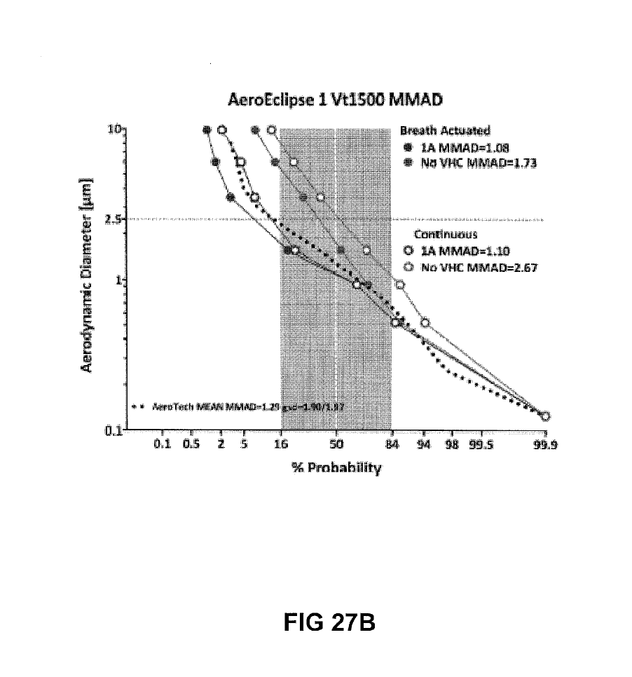

FIG. 27A shows the results with and without the chamber for the AeroEclipse jet nebulizer using the “COPD” tidal volume of 450 mL. The “standing cloud” indicates no ventilation and shows a curve to the right of the other curves (solid black), indicating large particles. The dotted line represents the best jet nebulizer tested in our lab, the AeroTech II (without any chamber). Whether breath actuated or continuous, the use of the chamber moves the curve to the left, indicating smaller particle sizes.

FIG. 27B shows the results with and without the chamber for the AeroEclipse jet nebulizer using the “Slow and Deep” pattern (tidal volume of 1.5 liters). The dotted line represents the best jet nebulizer tested in our lab, the AeroTech II (without any chamber). Whether breath actuated or continuous, the use of the chamber moves the curve to the left, indicating smaller particle sizes.

FIGS. 27A and B show that, with the chamber, we obtain excellent respirable aerosols (similar to dashed curve for AeroTech II) for both the COPD and slow and deep breathing patterns with virtually identical delivery. This shows that the same observations made on vibrating systems apply to nebulizers generally.

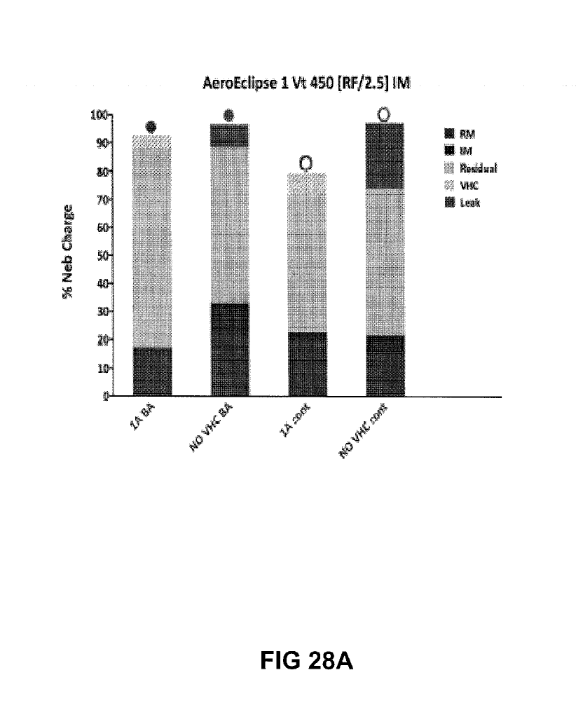

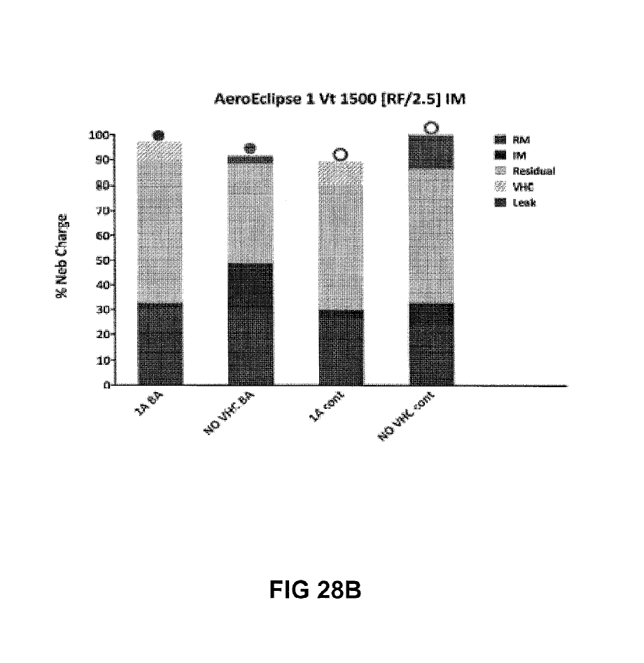

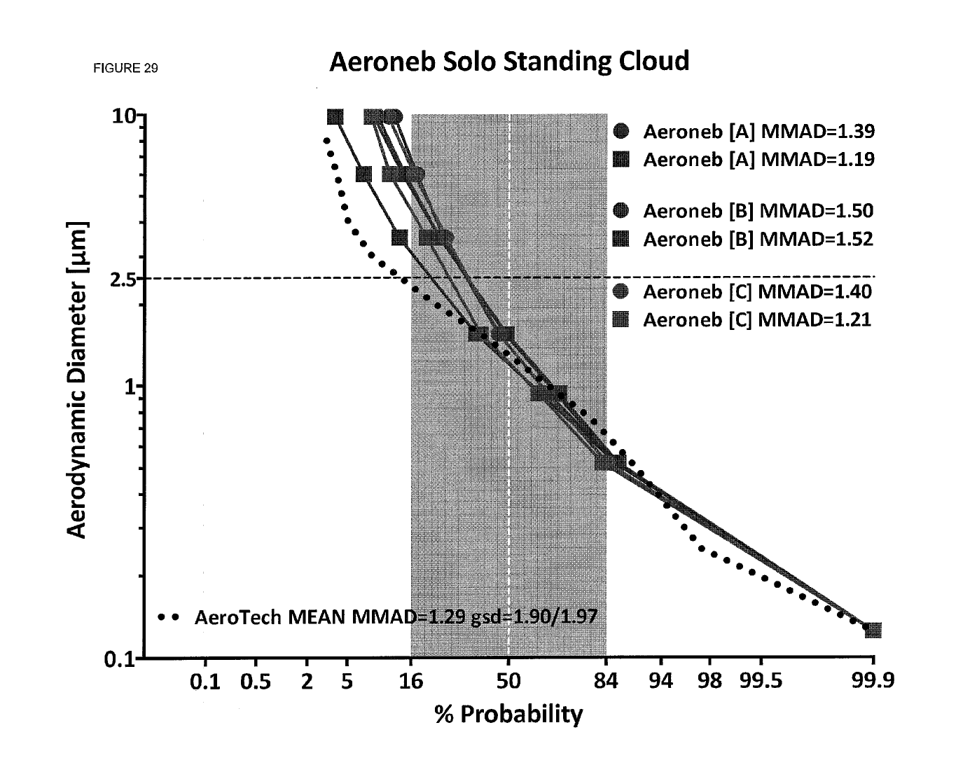

FIGS. 28 A and B are bar graphs depicting Inhaled Mass (IM) presented as a percent of the initial nebulizer charge. Light red bars indicate aerosol that will go to the lungs, dark red bars aerosols that will deposit in upper airways. Residuals are high because this is a jet nebulizer. The ‘leak’ represents aerosol that is not inhaled and lost during expiration. Again one can see that, whether using breath actuated (“BA”) or continuously breathing, excellent respirable aerosols are achieved. However, the data in Table 2 shows that treatment time when run continuously is reduced by as much as ½ when compared to that of BA (see Table 2, first column).

### Example 5

FIG. 29 depicts standing cloud distributions of a refined vibrating membrane system produced by the commercially available Aerogen Solo device. A-B-C represents 3 different membranes with different size holes; the circles and squares represent the results from two runs for each membrane. The membranes for this device are produced with hole distributions much smaller than those commonly available on the market for other nebulizers. For the standing cloud data, the MMAD range from 1.19 to 1.52 (much smaller than shown for the Omron on FIG. 18 (5.23-9.98). However, even these refined membranes still produce significant numbers of particles expected to deposit in the upper airways (approx. 20-30%).

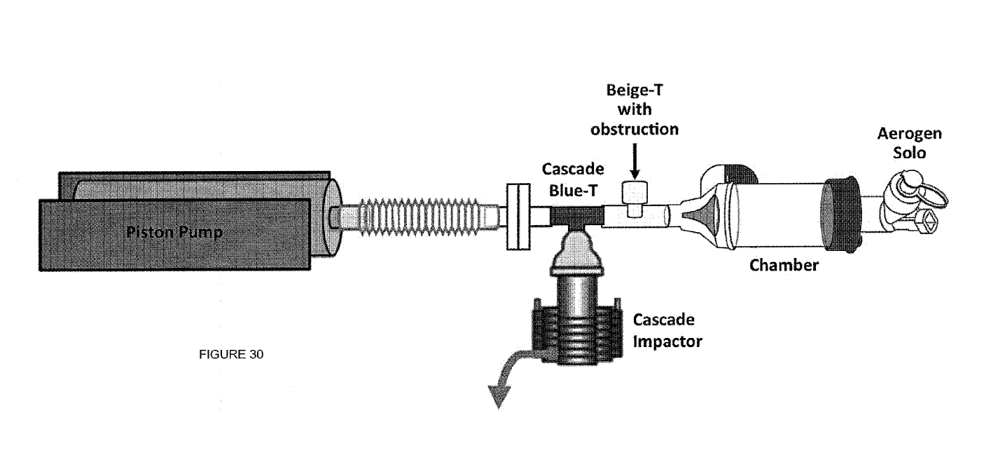

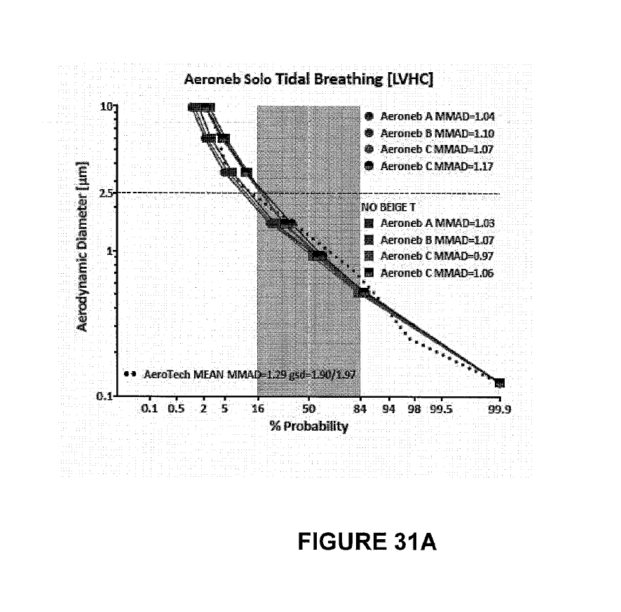

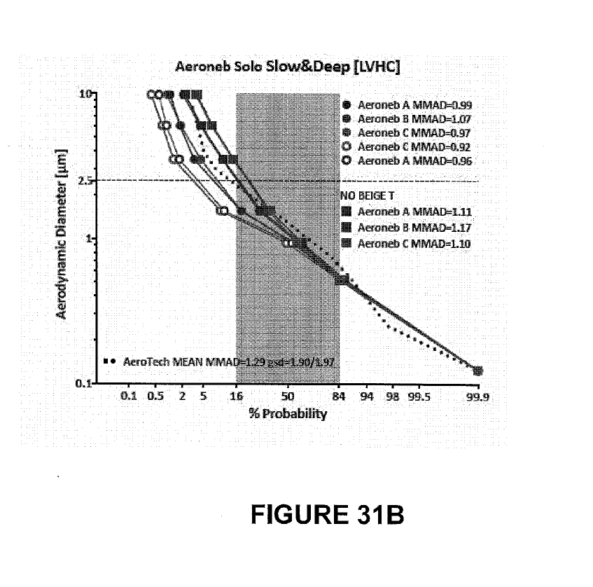

Addition of the chamber improves the distributions (approaching the dotted line which represents the results for the best jet nebulizer tested in our lab, the AeroTech II); however, the distributions can still lie to the right of the desired dotted distribution. To selectively remove these large particles, a narrowing tube or stenosis (i.e. constriction) was placed in the distal tubing from the chamber designed to remove by impaction particles primarily above 2.5 microns. The location of the stenosis is shown in FIG. 30 as “beige T.” While FIG. 30 shows a tube with a T shape, it need not be a T at all—but could just be a straight narrow tube with an obstruction. Addition of the stenosis (to the chamber) removes all the large particles from the distributions (as shown for the so called beige T data on FIGS. 31A and B, circles on the figure compared to squares).

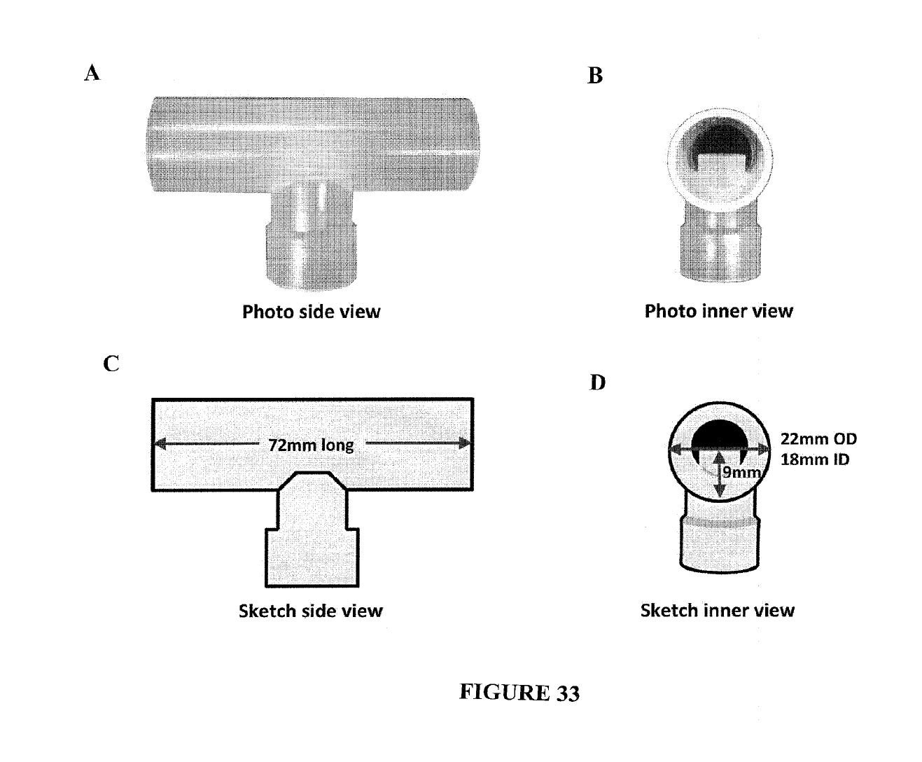

In these experiments, the vertical tube of the T was blocked, so the flow goes through the horizontal portion of the narrowing tube. Moreover, the narrowing tube contained an obstruction or baffle that projects into the lumen of the narrowing tube (see FIG. 33). While not limited to any precise mechanism, it is believed that the obstruction acts as a disrupter of the flow and either creates local turbulence or the particles directly impact. Indeed, both mechanisms are possible. The data on the figure “NO BEIGE T” were obtained with the entire T structure removed so that the particles could pass through without being obstructed by anything projecting into the lumen. Viewed in this light, the data with and without the beige T could be interpreted as with and without the projecting obstruction or baffle.

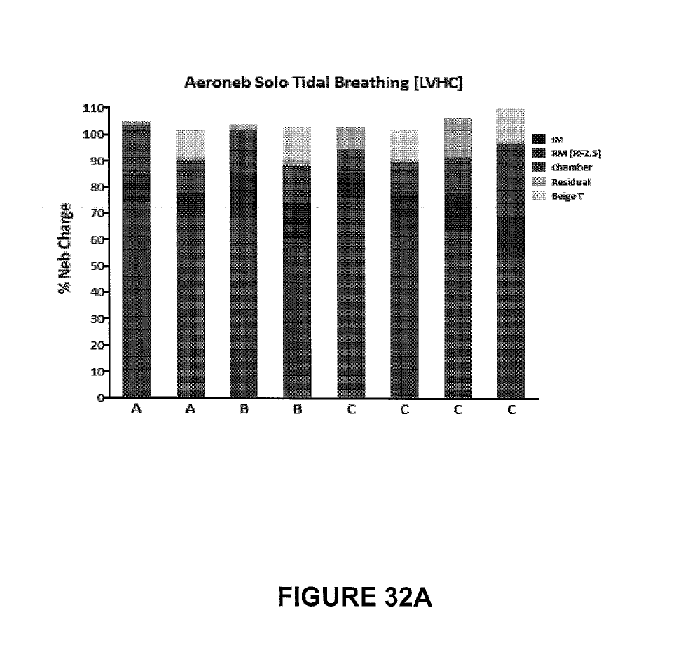

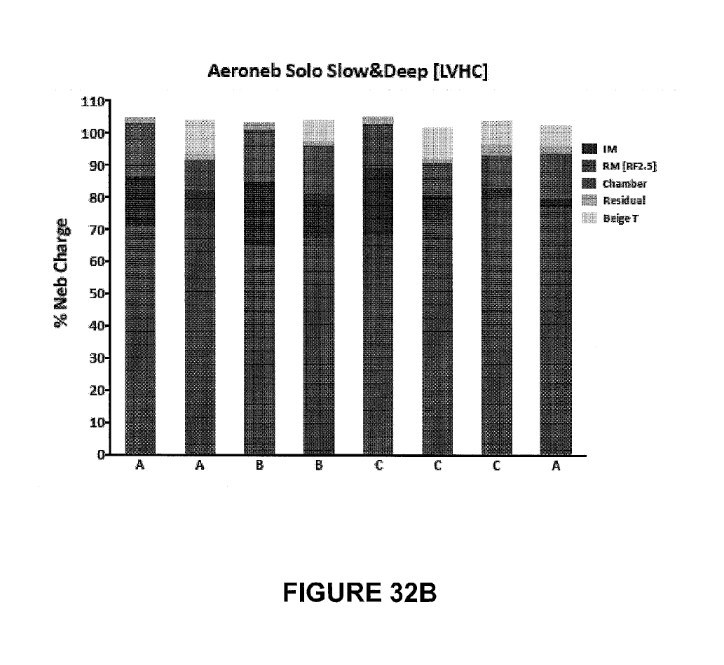

The bar graphs of FIGS. 32A and B indicate that ideal aerosols are obtained superimposed on or to the left of the desired dotted curve (particularly for slow and deep breathing) Again, A-B-C represents the same 3 membranes; squares are runs during breathing without the stenosis (runs without the beige T), circles are runs with the stenosis (with the beige T). It should be noted that all circle points are to the left of square points (better aerosols). Several runs were performed with the 3 membranes.

TABLE 1
Summary of in vitro data

| | | | | | MEAN ± SD | | | |
|---|---|---|---|---|---|---|---|---|
| | | | MEAN ± SD | 95% CI | | | Mean Tx | |
| BP | Description | Mode | IM* | | RF† | MMAD | Time [sec] | N |
| N/A | Standing Cloud | N/A | N/A | N/A | 0.180 ± 0.0776 | 6.74 ± 2.13 | N/A | 3 |
| COPD | No Chamber | Continuous | 31.0 ± 2.18 | 25.6-36.4 | 0.676 ± 0.158 | 1.77 ± 0.619 | 212 ± 175 | 3 |
| [450/15/0.35] | | Breath | 62.5 ± 6.68 | 45.9-79.1 | N/A | N/A | 545 ± 363 | 3 |
| | | Actuated | | | | | | |
| | Chamber | Continuous | 55.6 ± 8.35 | 34.9-76.3 | 0.822 ± 0.0719 | 1.27 ± 0.0907 | 212 ± 175 | 3 |
| | Chamber RH32% | | 60.9 | NA | 0.854 | 1.18 | | 1 |
| | Chamber RH50% | | 64.5 | NA | 0.816 | 1.27 | | 1 |
| | Chamber RH90% | | 62.2 | NA | 0.730 | 1.45 | | 1 |
| SLOW & DEEP | No Chamber | Continuous | 58.7 ± 11.0 | 31.3-86.0 | 0.538 ± 0.227 | 2.75 ± 1.77 | 212 ± 175 | 3 |
| [1500/5/0.7] | | Breath | 78.4 ± 1.99 | 73.4-83.3 | N/A | N/A | 319 ± 203 | 3 |
| | | Actuated | | | | | | |
| | Chamber | Continuous | 59.0 ± 4.31 | 43.3-69.7 | 0.770 ± 0.0753 | 1.49 ± 0.155 | 212 ± 175 | 3 |

*% of neb charge
†RF = Respirable fraction

TABLE 2

| Run Time | Vol | | | | | | | | | |
|---|---|---|---|---|---|---|---|---|---|---|
| [min] | [mL] | IM | RF | RM | Residual | VHC | Leak | RECOVERY | VHC type | MODE |
| 12.0 ± 0.7 | 3 | 17.5 | 0.8431 | 14.8 | 70.2 | 4.9 | | 93.4 | 1A/orig base | BA |
| 8.0 ± 0.0 | 3 | 22.7 | 0.8343 | 18.9 | 48.9 | 8 | | 79.6 | 1A/orig base | Cont. |
| 8.5 ± 0.7 | 3 | 21.5 | 0.741 | 15.9 | 52.3 | | 23.1 | 96.9 | NO VHC | Cont. |
| 15.0 ± 1.0 | 3 | 33.1 | 0.6732 | 22.283 | 55.3 | | 8.2 | 96.5 | NO VHC | BA |
| 24.0 ± 0.0 | 3 | 32.8 | 0.922 | 30.2 | 56.5 | 7.9 | | 97.2 | 1A/orig base | BA |
| 12.0 ± 0.0 | 3 | 30.1 | 0.8734 | 26.3 | 50.5 | 8.7 | | 89.3 | 1A/orig base | cont. |
| 12.5 ± 0.7 | 3 | 32.9 | 0.7218 | 23.7 | 53.3 | | 14.5 | 100.7 | No VHC | cont. |
| 24.5 ± 1.3 | 3 | 48.8 | 0.6122 | 29.9 | 39.5 | | 3.2 | 91.6 | No VHC | BA |

1-9. (canceled)

10. A method of capturing aerosol, comprising:
1) providing i) an aerosol generator, and ii) an aerosol capture device, said device comprising: a) an opening configured to connect to said aerosol generator, b) a chamber configured to capture all emitted aerosol particles from an aerosol generator when an operating aerosol generator is connected to said opening, said chamber comprising walls and a top and bottom, said bottom in fluid communication with said opening, said top comprising a one-way valve in fluid communication with, c) inhalation and exhalation openings, said inhalation opening comprising a mouthpiece;
2) connecting said aerosol generator to said aerosol capture device through said opening; and
3) operating said aerosol generator under conditions such that said chamber capture all emitted aerosol particles from said aerosol generator, wherein at least a portion of said particles contact said chamber, such that there is impaction and deposition on the walls, and said particles are mixed with air so as to reduce particle size such that the majority of aerosol particles are less than 2.5 microns in diameter.

11. The method according to claim 10, wherein said aerosol generator comprises a vibrating nebulizer.

12. The method according to claim 10, wherein said aerosol generator comprises a jet nebulizer.

13. The method according to claim 10, wherein said aerosol generator is a vibrating membrane.

14. The method according to claim 13, wherein a narrowing tube or stenosis is connected to said chamber at the end of the chamber opposite the aerosol generator.

15. An apparatus comprising an aerosol generator attached to an aerosol capture device, said aerosol generator comprising a vibrating mesh, said mesh comprising holes of less than 3.5 microns in diameter, said capture device comprising a) an opening connected to and in fluid communication with said aerosol generator, b) a chamber configured to capture all emitted aerosol particles from said aerosol generator when said aerosol generator is operating, said chamber comprising a top and bottom, said bottom in fluid communication with said opening, said top comprising a narrowing tube and a one-way valve, said narrowing tube positioned opposite the aerosol generator, said one-way valve in fluid communication with, c) inhalation and exhalation openings, said inhalation opening comprising a mouthpiece.

16. The apparatus according to claim 15, wherein said chamber comprises anti-static plastic.

17. The apparatus according to claim 15, wherein said aerosol generator comprises a vibrating membrane.

18-26. (canceled)

27. An apparatus comprising an aerosol generator comprising a vibrating element, said vibrating element located at the entrance of a chamber and comprising mesh, said mesh comprising holes less than 3.5 microns in diameter, said chamber configured to capture all emitted aerosol particles from said aerosol generator when said aerosol generator is operating, said chamber comprising an exit, said exit opposite said aerosol generator and comprising a narrowing tube and a one-way valve in fluid communication with, c) inhalation and exhalation openings, said inhalation opening comprising a mouthpiece.

28. The apparatus according to claim 27, wherein said vibrating element serves as the floor of the chamber.

29. The apparatus according to claim 27, wherein the aerosol generator is directly attached to the chamber.

30. The apparatus according to claim 27, wherein the aerosol generator is irreversible attached to the chamber.

31. The apparatus according to claim 27, wherein said chamber comprises anti-static plastic.

32. (canceled)

33. A method of administrating an aerosol, comprising:
a) providing an aerosol capture device, said capture device comprising a) an aerosol generator comprising a vibrating mesh, said mesh comprising holes of less than 4.0 microns in diameter, said aerosol generator positioned on the floor of b) a chamber comprising one or more walls and configured to capture all emitted aerosol particles from said aerosol generator when said aerosol generator is operating, said chamber comprising a top, sides and said floor, said top comprising a one-way valve in fluid communication with c) at least one opening for contacting a subject, said floor comprising d) an opening for introducing air into said chamber; and
b) activating said aerosol generator under conditions wherein i) said chamber captures all emitted aerosol particles from said aerosol generator, and mixes said particles with air, ii) at least a portion of said aerosol particles contact said chamber, such that there is impaction and deposition on the walls, iii) at least a portion of said aerosol particles leave said chamber when said subject inhales on said mouthpiece, and iii) said one-way valve blocks gases from entering said top of said chamber when said subject exhales.

34. The method of claim 33, wherein said mixing of said particles with air reduces the particle sizes of a plurality of particles.

35. The method of claim 33, wherein said mixing of said particles with air reduces the particle sizes such that the majority of aerosol particles are less than 2.5 microns in diameter

36. The method of claim 33, wherein said chamber causes impaction of the majority of said particles on said chamber.

37. The method of claim 33, wherein a narrowing tube connected to said chamber at said inhalation opening.

38. The method of claim 37, wherein said narrowing tube comprises an obstruction positioned therein.

39. The method of claim 37, wherein said narrowing tube comprises a baffle positioned therein.
